(19) United States
(12) Patent Application Publication
(10) Pub. No.: US 2014/0148390 A1
(43) Pub. Date: May 29, 2014

(54) FUSION PROTEINS RELEASING RELAXIN AND USES THEREOF

(71) Applicants: Ulrich Haupts, Odenthal (DE); Andreas Wilmen, Köln (DE)

(72) Inventors: Ulrich Haupts, Odenthal (DE); Andreas Wilmen, Köln (DE)

(73) Assignee: BAYER INTELLECTUAL PROPERTY GMBH, Monheim (DE)

(21) Appl. No.: 14/131,643

(22) PCT Filed: Jul. 4, 2012
(86) PCT No.: PCT/EP2012/062956

§ 371 Date: Jan. 8, 2014

(30) Foreign Application Priority Data
Jul. 8, 2011 (EP) 11173328.3

(51) Int. Cl. C07K 14/64; C07K 16/18

(52) U.S. Cl. CPC C07K 14/64; C07K 16/18
USPC 514/12.7; 530/399; 530/387.3; 530/363; 530/394; 536/23.4; 435/320.1; 435/252.3; 435/69.4; 435/252.33; 435/254.2; 435/254.21; 435/254.11; 435/419; 435/348; 435/369; 435/360; 435/365.1; 435/352; 435/367

(57) ABSTRACT

The present invention provides Relaxin fusion proteins, wherein a linker connects the carboxy-terminus of Relaxin with a proteinaceous half-life extending moiety and the linker comprises a protease cleavage site. Therefore, the invention provides Relaxin fusion polypeptides with extended half-life whereby the fusion protein by itself serves as a depot for release of the biologically active Relaxin. Furthermore, the invention provides nucleic acid sequences encoding the foregoing fusion polypeptides, vectors containing the same, cells expressing the Relaxin fusion polypeptides, pharmaceutical compositions and medical use of such fusion polypeptides.

The present invention provides Relaxin fusion proteins, wherein a linker connects the carboxy-terminus of Relaxin with a proteinaceous half-life extending moiety and the linker comprises a protease cleavage site. Therefore, the invention provides Relaxin fusion polypeptides with extended half-life whereby the fusion protein by itself serves as a depot for release of the biologically active Relaxin. Furthermore, the invention provides nucleic acid sequences encoding the foregoing fusion polypeptides, vectors containing the same, cells expressing the Relaxin fusion polypeptides, pharmaceutical compositions and medical use of such fusion polypeptides.

### BACKGROUND OF THE INVENTION

Relaxin 2 (H2 relaxin, RLN2) as a member of the insulin superfamily is a 2-chain peptide exhibiting, on the genetic level, the typical B-C-A chain prohormone structure, arranged from N- to C-terminus. Other members of this superfamily, encoded by 7 genes in human, are the relaxin genes RLN 1, RLN3, and the insulin-like peptide genes INSL3, INSL4, INSL5, and INSL6. The overall sequence homology between members of this family is low; nevertheless, phylogenetic analysis indicates that these genes have evolved from the RLN3 ancestral gene (Hsu, S. Y. (2003); Wilkinson, T. N. et al. (2005)). The mature protein has a molecular weight of approximately 6000 Da and is the product of an enzymatic cleavage of the prohormone catalyzed by the Prohormone-Convertase 1 (PC1) and 2 (PC2) (Hudson P. et al. (1983)). The resulting A- and B-chains are joined by two intermolecular cysteine bridges; the A-chain exhibits an additional intramolecular disulfide bond. Relaxin initiates pleiotropic effects through multiple pathways on a variety of cell types. It confers its activity by binding to the class I (rhodopsin like) G-protein-coupled receptor termed LGR7 (leucine-rich G protein-coupled receptor 7) also named RXFP1 (relaxin family peptide 1 receptor), and with significantly lower affinity to LRG8/RXFP2 (relaxin family peptide 2 receptor) (Kong R C et al. (2010)). Within the Relaxin molecule, an amino acid motif in the B-chain (Arg-X-X-X-Arg-X-X-Ile/Val-X) (Schwabe and Büllesbach (2007), Büllesbach and Schwabe (2000)) is conserved in all of the Relaxin peptides and is crucial for the interaction of these peptides with the corresponding receptor. Binding of Relaxin to LGR7/RXFP1 leads to activation of adenylate cyclase and to an increase of the second messenger molecule cAMP. Via this mechanism. Relaxin 2 for example mediates the release of atrial natriuretic peptide in rat hearts (Toth, M. et al. (1996)). A positive inotropic effect of Relaxin 2 on rat atrial myocytes has also been shown (Piedras-Renteria, E. S. et al. (1997)). Other signal transduction molecules which are activated by the Relaxin/LGR7 complex are the phosphoinositide-3 kinase, tyrosine kinases, and phosphodiesterases (Bartsch, O. et al. (2001), Bartsch, O. et al. (2004)). Additional signal transduction pathways activated by this system include the nitric oxide (NO) pathway leading to increased levels of cyclic GMP in rat and guinea-pig hearts (Bani-Sacchi, T. et al. (1995)).

Relaxin acts as a pleiotropic hormone (Dschietzig T. et al. (2006)) possessing biological activity on organs such as lung, kidney, brain, and heart. A strong antifibrotic and vasodilator activity of Relaxin is most notably responsible for the positive effects obtained with this peptide in various animal disease models as well as in clinical studies (McGuane J. T. et al. (2005)). RLN2 has multiple beneficial effects in the cardiovascular system under pathological conditions. It maintains tissue homeostasis and protects the injured myocardium during various pathophysiological processes. It exhibits prominent vasodilatory effects, e.g. affecting flow and vasodilation in rodent coronary arteries (Nistri. S. et al. (2003)) and in the vascular beds of other organs. In spontaneously hypertensive rats RLN2 lowered blood pressure, an effect mediated by increased NO production.

A cardioprotective activity of Relaxin 2 has been evaluated in different animal models such as guinea pig, rat and pig (Perna A. M. et al. (2005), Bani, D. et al. (1998)). RLN2 ameliorates myocardial injury, inflammatory cell infiltration and subsequent fibrosis, thereby alleviating severe ventricular dysfunction (Zhang J. et al. (2005)). Relaxin 2 exhibits strong antifibrotic activity. In injured tissues, fibroblast activation and proliferation causes increased collagen production and interstitial fibrosis. Fibrosis in the heart is increased by biomechanical overload, and influences ventricular dysfunction, remodeling, and arrhythmogenesis. In animal models, continuous infusion of Relaxin 2 inhibits or even reverses cardiac dysfunction caused by cardiomyopathy, hypertension, isoprenaline-induced cardiac toxicity, diabetic cardiomyopathy and myocardial infarction. This inhibition of fibrogenesis or reversal of established fibrosis can reduce ventricular stiffening and improve diastolic function. Notably, although Relaxin 2 reduces aberrant collagen accumulation, it does not affect basal collagen content in healthy tissues, highlighting its safety for therapeutic use.

Relaxin 2 has been tested in several clinical studies as a pleiotropic vasodilator for the treatment of patients with acute heart failure with very promising outcome. In these studies. Relaxin 2 was associated with favourable relief of dyspnoea and other clinical outcomes (Teerlink J. R. et al. (2009), Metra M. et al. (2010)) Due to the limited in-vivo half life of Relaxin, treatment of patients has to be repeated every 14 to 21 days, whereby compound administration has to be performed as a continuous infusion for at least 48 hours.

Furthermore. Relaxin 2 may also be useful in the treatment of diseases such as pancreatitis, inflammation-related diseases like rheumatoid arthritis, and cancer (Cosen-Binker L. I. et al. (2006) Santora K. Et al. (2007)) or scleroderma, pulmonary, renal, and hepatic fibrosis (Bennett R G. (2009)). Relaxin 2 reduces xenograft tumour growth of human MDA-MB-231 breast cancer cells (Radestock Y. Hoang-Vu C, Hombach-Klonisch S. (2008)).

The synthesis of Relaxin 2 by chemical methods is difficult. Due to the low solubility of the B-chain and the requirement for the laborious, specific introduction of cysteine bridges between A and B-chains, yields of active peptide obtained by these methods are extremely low (Barlos K. K. et al. (2010)). Alternatively, recombinant expression of Relaxin 2 can be performed. To allow efficient cleavage of the prepro-peptide during post-translational modifications and the secretion of mature and biological active peptides, expression host cells are routinely co-transfected with expression constructs encoding the Prohormone-Convertase 1 and/or 2 (Park J. I. et al. (2008)). Nevertheless, the endoproteolytic processing efficiency of prepro-peptides in heterologous cells often limits the production of bioactive molecules significantly (Shaw J. A. et al. (2002)).

Importantly, the half-life of intravenously administrated Relaxin 2 in humans is less than 10 minutes (Dschietzig T. et al. (2009)). As a consequence, in clinical trials Relaxin 2 has to be administered continuously over 48 h. Therefore, the improvement of the biological half life of Relaxin or longer acting Relaxin fusion polypeptides could be of great advantage.

Improving biological half life can either be performed by chemical modification such as PEGylation or HESylation of the polypeptide of interest, introduction of additional, non-natural N-glycosylation sites or by genetically fusing this polypeptide with other molecules such as the immunoglobulin Fc fragment of antibodies, transferrin, albumin, binding modules that bind in-vivo to other molecules mediating longer half-life, or other proteins, respectively. However, fusion of the Fc domain of an IgG to the C-terminus of Relaxin 2 leads to an inactive molecule with respect to the Relaxin activity. Surprisingly, it was found that when the Fc domain is cleaved off. Relaxin activity is regained. This implies that despite the inactivity of the fusion protein. Relaxin is correctly folded but activity is blocked by the Fc domain or Relaxin regains correct folding after release of the Fc domain. Fc fusion polypeptides for anti-complement pro drugs are disclosed in J Biol. Chem. 2003 Sep. 19; 278(38):36068-76. Therefore, the invention provides Relaxin fusion polypeptides where Relaxin is fused to proteinaceous half-life extending moieties such as a Fc domain of an IgG wherein the Relaxin is linked to the proteinaceous half-life extending moiety via a linker polypeptide comprising an endo-protease cleavage site, leading to a polypeptide with improved half-life compared to Relaxin, from which active Relaxin is released by the action of an endoprotease.

### SUMMARY OF THE INVENTION

The invention concerns half-life extended Relaxin fusion polypeptides as a pro-drug for the release of active Relaxin.

One embodiment of the invention is a fusion polypeptide comprising Relaxin, a linker peptide comprising an endo-protease cleavage site and a proteinaceous half-life extending moiety, wherein the linker peptide connects Relaxin with the half-life extending moiety.

In one embodiment the aforementioned Relaxin is a Relaxin 2 or a Relaxin 3. Preferred is human Relaxin, such as human Relaxin 2 or human Relaxin 3.

In one embodiment the aforementioned proteinaceous half-life extending moiety is a polypeptide, such as Fc domain of an IgG, serum albumin, transferrin, or a serum albumin binding protein or peptide. Preferred is a human or humanized proteinaceous half-life extending moiety such as the Fc domain of an human IgG or human serum albumin.

In a preferred embodiment the aforementioned linker comprises a cleavage site for an endo-protease/endo-peptidase, wherein the endo-protease/endo-peptidase is an extra-cellular endo-protease/endo-peptidase. In a further preferred embodiment the aforementioned linker comprises a cleavage site for an endo-protease/endo-peptidase, wherein the endo-protease/endo-peptidase is a human endo-protease/endo-peptidase. In a further preferred embodiment the cleavage site is of an endo-protease/endo-peptidase which is active in blood such as blood coagulation factor Xa. Additionally, the cleavage site of a membrane-bound or membrane stretching endo-protease/endo-peptidase which has active sites that are directed towards the lumen of blood vessels are preferred, such as MMP12. In another preferred embodiment the cleavage site is of an endo-protease/endo-peptidase the activity of which is enriched or specific at sites where the action of Relaxin is desired, e.g. the endo-protease/endo-peptidase is specifically expressed and/or activated at the site of desired Relaxin activity such as specific organs or tissues. In another preferred embodiment the cleavage site is of an endo-protease/endo-peptidase which is expressed and/or activated at specific time points during physiologic processes, e.g. at specific time points of the development of a disease.

In another aspect, the invention provides a polynucleotide encoding an aforementioned fusion polypeptide. Such a polynucleotide may further comprise a coding sequence for a signal peptide allowing secretion of the fusion polypeptide. Vectors containing polynucleotides for such fusion polypeptides are included as well. Suitable vectors are for example expression vectors. A further embodiment of the invention is a host cell comprising a polynucleotide, a vector, or expression vector encoding the aforementioned fusion polypeptides. The host cell of the invention can be an eukaryotic cell or a prokaryotic cell. An eukaryotic cell can be a mammalian cell or a yeast or insect cell, preferably a mammalian cell. A prokaryotic cell can be for example an E. coli cell.

In another embodiment the invention provides pharmaceutical compositions comprising the aforementioned fusion polypeptides. The composition may be formulated for intravenous, intraperitoneal, topical, inhalative or subcutaneous administration.

Another embodiment of the invention provides a pharmaceutical composition or a fusion polypeptide as medicament. A further embodiment is the use of a pharmaceutical composition or a fusion polypeptide in the treatment of cardiovascular diseases, pancreatitis, inflammation, cancer, scleroderma, pulmonary, renal, and hepatic fibrosis.

### BRIEF DESCRIPTION OF THE DRAWINGS

FIG. 1 Schematic representation of the organization of a Relaxin fusion polypeptide and its subsequent activation in the blood stream by an endo-peptidase/endo-protease cleaving the linker comprising a Protease Cleavage Site (PCS). A-chain, B-chain and C-chain represent the respective Relaxin chains. Linker with PCS is a linker comprising a PCS and black lines denote inter- and intramolecular disulfide bonds in Relaxin. Fc domain is a Fc domain of an IgG molecule.

FIG. 2 Determination of the activity of the Relaxin-Fc fusion construct using the CHO—CRE-LGR7 cell line. As control, hRelaxin 2 (R&D Systems, catalogue number 6586-RN-025) was used. Data are expressed as Relative Light Units, representing the activity of the Relaxin variants and hRelaxin 2 induced luciferase expression. Symbols represent means, error bars represent S.E.M.

FIG. 3 a-d Determination of the activity of the Relaxin-Fusion constructs 1-4 using the CHO-CRE-LGR7 cell line. As control, hRelaxin 2 (R&D Systems, catalogue number 6586-RN-025) was used. Data are expressed as Relative Light Units, representing the activity of the Relaxin variants and hRelaxin 2 induced luciferase expression. Symbols represent means, error bars represent S.E.M.

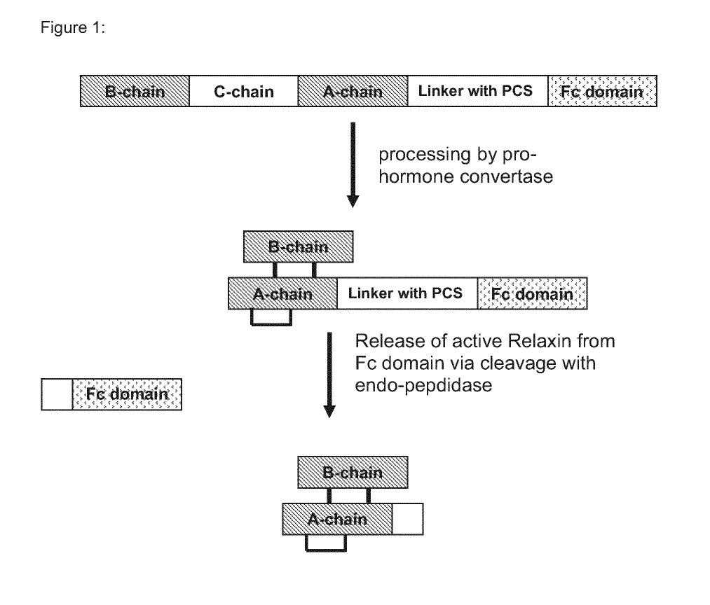

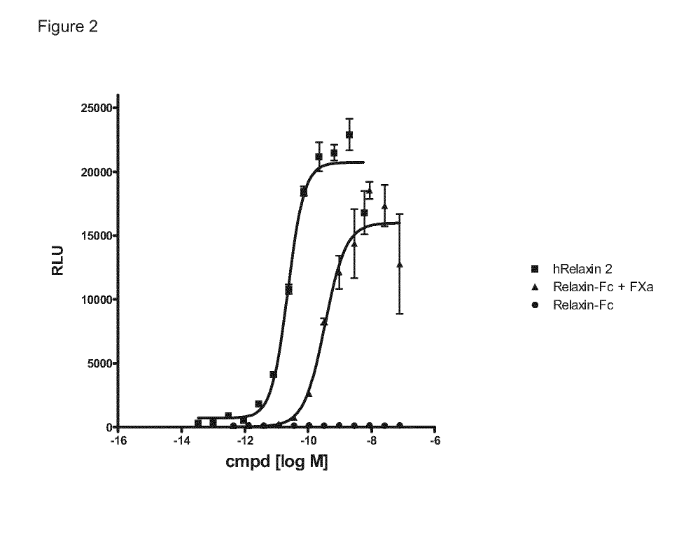

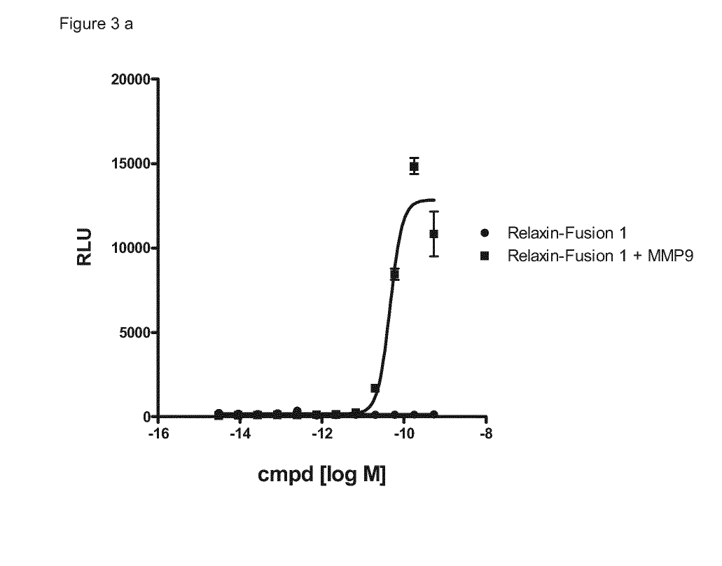

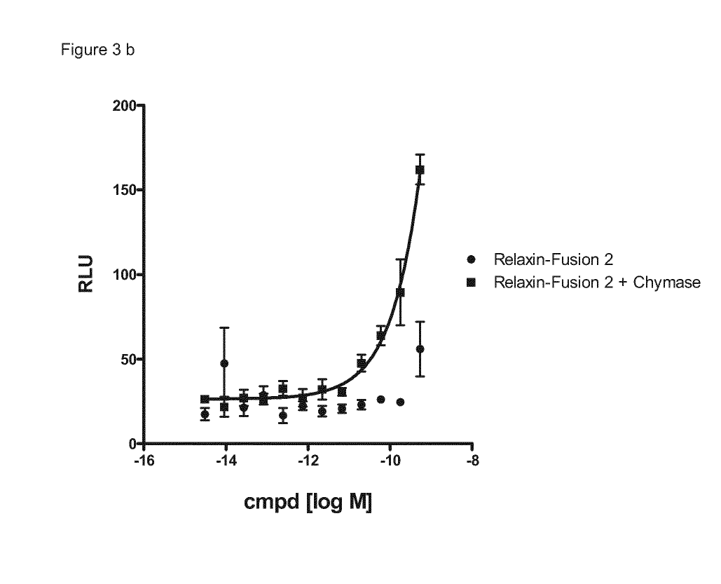

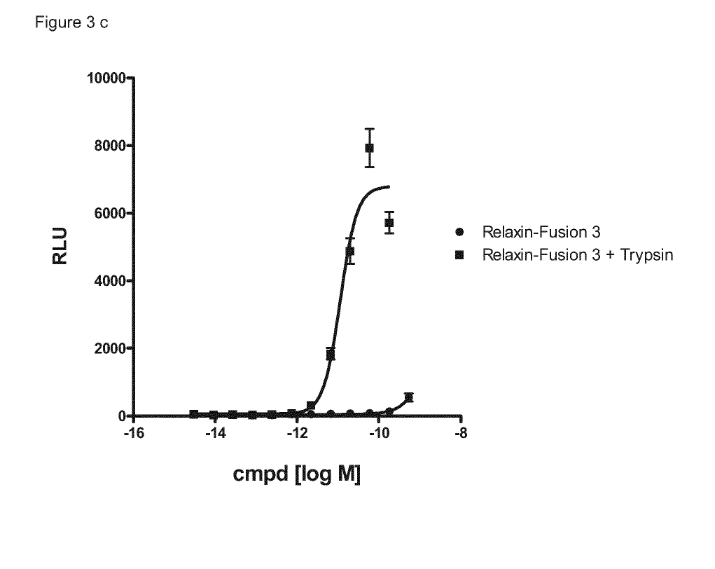

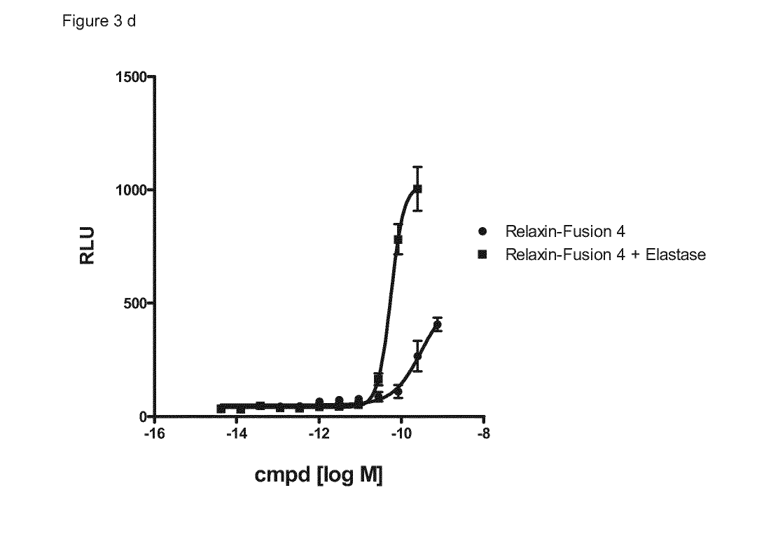

### DETAILED DESCRIPTION OF THE INVENTION

### Definitions

The term “amino acid residue” is intended to indicate an amino acid residue contained in the group consisting of alanine (Ala or A), cysteine (Cys or C), aspartic acid (Asp or D), glutamic acid (Glu or E), phenylalanine (Phe or F), glycine (Gly or G), histidine (H is or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or 5), threonine (Thr or T), valine (Val or V), tryptophan (Trp or W), and tyrosine (Tyr or Y) residues.

The term “activity of Relaxin” or “Relaxin Acitvity” is defined by the ability of Relaxin or variants thereof to activate the stimulatory G-protein Gs through binding to its receptors and thus the subsequent generation of the second messenger cyclic AMP, and/or the stimulation of PI3-kinase. Relaxin or variants thereof bind to LGR7 leading to the intracellular activation of the stimulatory G-protein Gs, resulting in the subsequent generation of the second messenger cyclic AMP (cAMP). However, cAMP generation is a time-dependent biphasic response. After an initial short Gs-adenylate cyclase-mediated cAMP response the receptor signal is switching to an inhibitory G protein activation and by this to PI3-kinase-mediated response. (Halls M. L., Bathgate R. A., Summers, R. J. (2005)).

The term “half-life extending moiety” refers to a pharmaceutically acceptable moiety, domain, or “vehicle” covalently linked (“conjugated”) to the Relaxin fusion polypeptide directly or via a linker. Mechansims by which the half-life extending moiety positively influences pharmacokinetic or pharmacodynamic behaviour include but are not limited to (i) preventing or mitigating in vivo proteolytic degradation or other activity-diminishing chemical modification of the Relaxin fusion polypeptide, (ii) improving half-life or other pharmacokinetic properties by reducing renal filtration, decreasing receptor-mediated clearance or increasing bioavailability, (iii) reducing toxicity, (iv) improving solubility, (v) increasing biological activity and/or target selectivity of the Relaxin fusion polypeptide. In addition the half-life extending moiety may have positive effects on terms of increasing manufacturability, and/or reducing immunogenicity of the Relaxin fusion polypeptide, compared to an unconjugated form of the Relaxin fusion polypeptide. The term “half-life extending moiety” includes non-proteinaceous, half-life extending moieties, such as PEG or HES, and proteinaceous half-life extending moieties, such as serum albumin, transferrin or Fc domain.

“Polypeptide”, “peptide” and “protein” are used interchangeably herein and include a molecular chain of two or more amino acids linked through peptide bonds. The terms do not refer to a specific length of the chain. The terms include post-translational modifications of the polypeptide, for example, glycosylations, acetylations, phosphorylations and the like. In addition, protein fragments, analogs, mutated or variant proteins, fusion proteins and the like are included in the definition of polypeptide, peptide or protein. The terms also include molecules in which one or more amino acid analogs or non-canonical or unnatural amino acids are included as can be synthesized, or expressed recombinantly using known protein engineering techniques. In addition, inventive fusion proteins can be derivatized as described herein by well-known organic chemistry techniques.

The term “functional variant” refers to a variant polypeptide which differs in its chemical structure from the wild-type polypeptide and retains at least some of its natural biological activity. In case of the Relaxin 2 variants according to the invention, a functional variant is a variant which shows at least some of its natural activity, such as the activation of the relaxin receptor LGR7. The activation of the relaxin receptor LGR7 can be determined by a method disclosed in experimental methods.

The terms “fragment,” “variant,” “derivative,” and “analog” when referring to polypeptides of the present invention include any polypeptides that retain at least some of the receptor activating properties of the corresponding wild-type Relaxin polypeptide. Fragments of polypeptides of the present invention include proteolytic fragments, as well as deletion fragments, and also polypeptides with altered amino acid sequences due to amino acid substitutions, deletions, or insertions. Variants may occur naturally or be non-naturally occurring. Non-naturally occurring variants may be produced using mutagenesis techniques known in the art. Variant polypeptides may comprise conservative or non-conservative amino acid substitutions, deletions, or additions. Variant polypeptides may also be referred to herein as “polypeptide analogs.” As used herein a “derivative” of a polypeptide refers to a subject polypeptide having one or more residues chemically derivatized by reaction of a functional group. Also included as “derivatives” are those peptides that contain one or more naturally occurring amino acid derivatives of the twenty standard amino acids. For example, proline may be substituted by 4-hydroxyproline; lysine may be substituted by 5-hydroxylysine; histidine may be substituted by 3-methylhistidine; serine may be substituted by homoserine; and lysine may be substituted by ornithine.

The term “fusion protein” or “fusion polypeptide” indicates that the protein includes polypeptide components derived from more than one parental protein or polypeptide and/or that the fusion protein includes protein domains derived from one or more parental protein or polypeptides which are not arranged in their wild type orientation. Typically, a fusion protein is expressed from a fusion gene in which a nucleotide sequence encoding a polypeptide sequence from one protein is appended in frame with, and optionally separated by a linker or stretcher from, a nucleotide sequence encoding a polypeptide sequence from a different protein. The fusion gene can then be expressed by a recombinant host cell as a single protein.

The term “nucleotide sequence” or “polynucleotide” is intended to indicate a consecutive stretch of two or more nucleotide molecules. The nucleotide sequence may be of genomic, cDNA. RNA, semisynthetic, synthetic origin, or any combinations thereof.

The term “EC₅₀” (half maximal effective concentration) refers to the effective concentration of a therapeutic compound which induces a response halfway between the baseline and maximum under the specific experimental conditions. The term “immunogenicity” as used in connection with a given substance is intended to indicate the ability of the substance to induce a response of the immune system. The immune response may be a cell or antibody mediated response (see, e.g., Roitt: Essential Immunology (8th Edition, Black-well) for further definition of immunogenicity). Normally, reduced induction of processes involved in triggering an immune response such as T-cell proliferation will be an indication of reduced immunogenicity. The reduced immunogenicity may be determined by use of any suitable method known in the art, e.g. in vivo or in vitro.

The term “polymerase chain reaction” or “PCR” generally refers to a method for amplification of a desired nucleotide sequence in vitro, as described, for example, in U.S. Pat. No. 4,683,195 and U.S. Pat. No. 4,683,195. In general, the PCR method involves repeated cycles of primer extension synthesis, using oligonucleotide primers capable of hybridizing preferentially to a template nucleic acid.

The term “vector” refers to a plasmid or other nucleotide sequences that are capable of replicating within a host cell or being integrated into the host cell genome, and as such, are useful for performing different functions in conjunction with compatible host cells (a vector-host system): to facilitate the cloning of the nucleotide sequence, i.e. to produce usable quantities of the sequence, to direct the expression of the gene product encoded by the sequence and to integrate the nucleotide sequence into the genome of the host cell. The vector will contain different components depending upon the function it is to perform.

“Cell”, “host cell”, “cell line” and “cell culture” are used interchangeably herein and all such terms should be understood to include progeny resulting from growth or culturing of a cell.

The term “functional in vivo half-life” is used in its normal meaning, i.e. the time at which 50% of the biological activity of the polypeptide is still present in the body/target organ, or the time at which the activity of the polypeptide is 50% of the initial value.

As an alternative to determining functional in vivo half-life, “serum half-life” may be determined, i.e. the time at which 50% of the polypeptide circulates in the plasma or bloodstream prior to being cleared independent of whether the polypeptide retains its biological function. Determination of serum half-life is often easier than determining the functional in vivo half-life and the magnitude of serum half-life is usually a good indication of the magnitude of functional in vivo half-life. Alternative terms to serum half-life include “plasma half-life”, “circulating half-life”, “serum clearance”, “plasma clearance”, “terminal half-life” and “clearance half-life”. The polypeptide is cleared by the action of one or more of the reticuloendothelial systems (RES), kidney, spleen or liver, by tissue factor, SEC receptor or other receptor mediated elimination, or by specific or unspecific proteolysis. Normally, clearance depends on size (relative to the cutoff for glomerular filtration), charge, attached carbohydrate chains, and the presence of cellular receptors for the protein. The functionality to be retained is normally determined as receptor binding or receptor activation. The functional in vivo half-life and the serum half-life may be determined by any suitable method known in the art and may for example generally involve the steps of suitably administering to a mammalian a suitable dose of the protein or polypeptide of interest; collecting blood samples or other samples from said mammalian at regular intervals; determining the level or concentration of the protein or polypeptide of interest in said blood sample; and calculating, from (a plot of) the data thus obtained, the time until the level or concentration of the protein or polypeptide of interest has been reduced by 50% compared to the appropriate reference time point, e.g. intial concentration shortly after i.v. application. Reference is for example made to the standard handbooks, such as Kenneth. A et al: Chemical Stability of Pharmaceuticals: A Handbook for Pharmacists and in Peters et al. Pharmacokinete analysis: A Practical Approach (1996). Reference is also made to “Pharmacokinetics”, M Gibaldi and D Perron, published by Marcel Dekker, 2nd Rev. edition (1982).

“Glycosylation” is a chemical modification wherein sugar moieties are added to the polypeptide at specific sites. Glycosylation of polypeptides is typically either N-linked or O-linked. N-linked refers to the attachment of a carbohydrate moiety to the side chain of an asparagine residue. The tripeptide sequences Asn-X-Ser and Asn-X-Thr (“N-X-S/T”), where X is any amino acid except proline, are the recognition sequences for enzymatic attachment of the carbohydrate moiety to the asparagine side chain. Thus, the presence of either of these tripeptide sequences (or motifs) in a polypeptide creates a potential N-linked glycosylation site. O-linked refers to the attachment of a carbohydrate moiety to the hydroxyl-group oxygen of serine and threonine.

An “isolated” polypeptide or fusion polypeptide is one that has been identified and separated from a component of the cell that expressed it and/or the medium into which it was secreted. Contaminant components of the cell are materials that would interfere with diagnostic or therapeutic uses of the fusion polypeptide, and may include enzymes, hormones, and other proteinaceous or non-proteinaceous solutes. In preferred embodiments, the fusion polypeptide is purified (1) to greater than 95% by weight of fusion polypeptide as determined e.g. by the Lowry method, UV-Vis spectroscopy or by by SDS-Capillary Gel electrophoresis (for example on a Caliper LabChip GXII, GX 90 or Biorad Bioanalyzer device), and in further preferred embodiments more than 99% by weight, (2) to a degree sufficient to obtain at least 15 residues of N-terminal or internal amino acid sequence, or (3) to homogeneity by SDS-PAGE under reducing or non-reducing conditions using Coomassie blue or, preferably, silver stain. Ordinarily, however, isolated fusion polypeptides will be prepared by at least one purification step.

### Overview

The application provides a Relaxin fusion protein with extended half-life. The present application describes improved Relaxin fusion proteins with significantly elongated biological half-life and significantly reduced biological activity. Due to the fact, that Relaxin is connected to the half-life extending moiety by a stretch of amino acids encoding a cleavage site for a protease that is active in vivo and releases functional relaxin from the Relaxin fusion protein, this Relaxin fusion protein exhibits a pharmacological depot effect.

One embodiment of the invention is a fusion protein comprising Relaxin-PCS-HEM, wherein Relaxin is a Relaxin hetreodimer comprising the processed A and B chains or a functional variant thereof, PCS is a linker polypeptide comprising a protease cleavage site (PCS) and HEM is a proteinaceous half-life extending moiety (HEM).

A further embodiment of the invention is a fusion polypeptide comprising proRelaxin-PCS-HEM, wherein proRelaxin is an unprocessed proform of Relaxin still containing the C-chain or a functional variant thereof, PCS is a linker polypeptide comprising a protease cleavage site (PCS) and HEM is a proteinaceous half-life extending moiety (HEM).

Another embodiment of the invention is a fusion protein comprising HEM-PCS-Relaxin wherein Relaxin is a Relaxin hetreodimer comprising the processed A and B chain or a functional variant thereof. PCS is a linker polypeptide comprising a protease cleavage site (PCS) and HEM is a proteinaceous half-life extending moiety (HEM).

A further embodiment of the invention is a fusion protein comprising HEM-PCS-proRelaxin wherein proRelaxin is an unprocessed proform of Relaxin still containing the C-chain or a functional variant thereof. PCS is a linker polypeptide comprising a protease cleavage site (PCS) and HEM is a proteinaceous half-life extending moiety (HEM).

proRelaxin is understood as the proform of Relaxin which is not processed by a prohormone convertase and comprises the Relaxin B chain, the Relaxin C-chain and the Relaxin A-chain in its natural orientation.

Relaxin-PCS-HEM and proRelaxin-PCS-HEM are preferred embodiments.

### Relaxin Domain:

In a further embodiment the Relaxin comprises a Relaxin 2 A chain polypeptide or a functional variant thereof. In a further embodiment the Relaxin comprises a Relaxin 2 B chain polypeptide or a functional variant thereof.

In a further embodiment the Relaxin comprises a Relaxin 2 A chain polypeptide or a functional variant thereof and a Relaxin 2 B chain polypeptide or a functional variant thereof.

In a preferred embodiment the Relaxin A chain polypeptide comprises a human minimal Relaxin 2A chain polypeptide (SEQ ID NO: 7) or a functional variant thereof, or comprises a human Relaxin 2 A chain polypeptide (SEQ ID NO: 6) or a functional variant thereof. In a preferred embodiment the Relaxin B chain polypeptide comprises a human Relaxin 2 B chain polypeptide (SEQ ID NO: 8) or a functional variant thereof.

In a more preferred embodiment the Relaxin A chain comprises a human minimal Relaxin 2 A chain polypeptide (SEQ ID NO: 7) or a functional variant thereof, or comprises a human Relaxin 2 A chain polypeptide (SEQ ID NO: 6) or a functional variant thereof and the Relaxin B chain polypeptide comprises a human Relaxin 2 B chain polypeptide (SEQ ID NO: 8) or a functional variant thereof.

In a further embodiment the Relaxin comprises a Relaxin 3 A chain polypeptide or a functional variant thereof and/or a Relaxin 3 B chain polypeptide or a functional variant thereof.

In a further embodiment the Relaxin A chain comprises a human Relaxin 3 A chain polypeptide (SEQ ID NO:9), human minimal Relaxin 3 A chain polypeptide (SEQ ID NO:12), or a functional variant thereof. In a further embodiment the Relaxin B chain polypeptide comprises a human Relaxin 3 B chain polypeptide (SEQ ID NO: 11) or a functional variant thereof. In a preferred embodiment the Relaxin comprises a human Relaxin 3 A chain polypeptide (SEQ ID NO: 10) or a functional variant thereof and comprises a human Relaxin 3 B chain polypeptide (SEQ ID NO: 11) or a functional variant thereof.

In a preferred embodiment a functional variant of the Relaxin A or B chain has 1, 2, 3, 4, 5, 6, 7, 8, 9, or 10 amino acid substitutions, insertions and/or deletions compared to the wild type Relaxin A and B chain, respectively. Further preferred is an aforementioned Relaxin 2 B variant that further comprises the conserved motif Arg-X-X-X-Arg-X-X-Ile/Val-X where X represents amino acids which are able to form a helical structure.

Relaxin A and B chain variants are known in the art. The well characterized binding site geometry of Relaxin provides the skilled person with guidance to design Relaxin A and B chain variants, see for example Büllesbach and Schwabe J Biol. Chem. 2000 Nov. 10; 275(45):35276-80 for variations of the Relaxin B chain and Hossain et al. J Biol. Chem. 2008 Jun. 20; 283(25):17287-97 for variations of the Relaxin A chain and the “minimal” Relaxin A chain. For example, for the conserved Relaxin 2 B motif (Arg-X-X-X-Ile/Val-X) X represents amino acids which are able to form a helical structure example to select appropriate amino acids X in the conserved motif as the three defined amino acids form a receptor contact region on the surface of the Relaxin B chain (Büllesbach and Schwabe, (2000)).

In an even more preferred embodiment the Relaxin A chain polypeptide is a human Relaxin 2 A chain polypeptide (SEQ ID NO: 6) or a functional variant thereof and the Relaxin B chain polypeptide is a human Relaxin 2 B chain polypeptide (SEQ ID NO: 8) or a functional variant thereof. In a even more preferred embodiment, the functional variant of human Relaxin 2 A chain polypeptide (SEQ ID NO: 6) is a functional variant having 1, 2, 3, 4, 5, 6, 7, 8, 9, or 10 amino acid substitutions, deletions and/or insertions compared to SEQ ID NO: 16. Further preferred is a functional variant of human Relaxin 2 B chain polypeptide (SEQ ID NO: 8) wherein the functional variant has 1, 2, 3, 4, 5, 6, 7, 8, 9, or 10 amino acid substitutions, deletions and/or insertions compared to SEQ ID NO: 8. Even further preferred is an aforementioned human Relaxin 2 B variant that further comprises the conserved motif Arg-X-X-X-Arg-X-X-Ile/Val-X.

In an even more preferred embodiment the Relaxin A chain polypeptide is a human Relaxin 2 A chain polypeptide (SEQ ID NO: 6) or a functional variant thereof having 1, 2, 3, 4, 5, 6, 7, 8, 9, or 10 amino acid exchanges compared to SEQ ID NO: 6 and the Relaxin B chain polypeptide is a human Relaxin 2 B chain polypeptide (SEQ ID NO: 8) or a functional variant thereof having 1, 2, 3, 4, 5, 6, 7, 8, 9, or 10 amino acid exchanges compared to SEQ ID NO: 18 and comprising the conserved motif Arg-X-X-X-Arg-X-X-Ile/Val-X.

The person skilled in the art knows how to obtain functional variants. Examples of functional variants are disclosed for the Relaxin A chain in Hossain et al J Biol. Chem. 2008 Jun. 20; 283(25):17287-97 or in US Pat. publication No. US2011/0130332 and for the Relaxin B chain in Schwabe and Büllesbach (2007) Adv Exp Med. Biol. 612:14-25 and Büllesbach and Schwabe J Biol. Chem. 2000 Nov. 10; 275(45):35276-80).

### PCS Linker:

To release Relaxin from the fusion protein, the employed linker sequence PCS comprises a cleavage sequence for a protease/peptidase. Proteases/peptidases are a group of enzymes whose catalytic function is to hydrolyze (breakdown) peptide bonds of proteins. They are also called proteolytic enzymes or proteinases. Proteases differ in their ability to hydrolyze peptide bonds. i.e. proteases may have preference for a specific peptide sequence as recognition and cleavage site. Proteases are subdivided into six groups, whereas Serine proteases, such as coagulation factor IIa. VIIa, and Xa, and Metalloproteases, such as Matrix Metalloprotease 2 and 9, represent the largest families.

Cleavage site position of the protease substrate is designated P1-P1′, meaning that the amino acid at the N terminal site of the cleavage site is defined as P1 and at the C terminal site defined as P1′. Amino acids in the N-terminal direction of the cleaved peptide bond are numbered as P2. P3, and P4. On the carboxyl side of the cleavage site numbering is likewise incremented (P1′, P2′, P3′ etc.) (Schlechter and Berger (1967 and 1968)).

In the context of the present invention a protease/peptidase is an endoprotease/endopeptidase. Endopeptidase or endoproteases are proteolytic peptidases that break peptide bonds of non-terminal amino acids (i.e. within a protein). In contrast thereto are exopeptidases, which hydrolyze either N- or C-terminal peptide bonds and therefore release the N-terminal or C-terminal amino acid of a polypeptide. For this reason, endopeptidases which cleave the PCS linker can release Relaxin in a controlled manner form a pro drug fusion protein.

In a preferred embodiment the PCS is a PCS of an endo-protease. In a preferred embodiment the PCS is a PCS of an extracellular endo-protease. In further preferred embodiment the aforementioned endo-protease is active in blood or at sites in the body where the action of Relaxin is desired. Even more preferred are endo-proteases which naturally occur in blood, such as coagulation factor Xa or in a diseased tissue of a Relaxin treatable disease, such as MMP metallo-proteases. Also preferred are endo-proteases which are membrane bound or membrane spanning but having their catalytic domain hence their catalytic activity in the lumen of blood vessels (hence in human blood) or exposed to the interstitial space in tissues, such as MMP12. Even further preferred are aforementioned endo-proteases being active in human blood and/or a diseased tissue of a Relaxin treatable disease. A Relaxin treatable disease is for example a fibrotic disease. The diseased tissue of a fibrotic disease therefore is for example lung, heart, liver or kidney tissue. Further Relaxin treatable diseases are listed below. Most preferred are aforementioned endo-proteases being of human origin or humanized.

A person skilled in the art knows that according to the EC nomenclature endoproteases belong to the group of EC EC 3.4.21-EC 3.4.24 (determined by the Nomenclature Committee of the International Union of Biochemistry and Molecular Biology). Useful endoproteases are for example trypsin. Thrombin, factor Xa, factor VIIa, MMP2, MMP12 or Renin.

It is also contemplated that an exogenous endo-protease cleaving the PCS can be administered leading to a release of Relaxin from the pro-drug. In a preferred embodiment this endogeneous protease is targeted to the desired site of Relaxin activity (e.g. a diseased tissue of a Relaxin treatable disease) through a targeting moiety connected to the protease.

Knowledge about the expression of the aforementioned endoproteases is state of the art. In some aspects of the invention it is preferred not only having a Relaxin with longer half-life as a pro drug but also to have Relaxin released from the pro drug in a specific organ or part of the body. Therefore, one can make use of the information in the art where a endoprotease is expressed to tailor the site of release of Relaxin from the pro drug.

Having a systemic release of Relaxin from the pro drug one would choose an endo-protease being present in blood. Such a protease for example is coagulation factor Xa.

As Relaxin released from its pro drug has a short half life, tailoring Relaxin release in specific organs, tissues or compartments, especially diseased organs, tissues or compartments, further improve its pharmaceutical benefit as Relaxin is released at the site of disease.

For example, Relaxin has a direct anti-hypertrophic effect on cardiomyocytes and anti-fibrotic activity on cardiac fibroblasts (Moore X L. Et al. (2007); Wang P. et al. (2009)). Therefore, proteases are preferred which are expressed predominantly in cardiac tissue, such as MMP2 (Overall C M. (2004)) or Chymase (Matsumoto C. et al. (2009)). Other prominent organs effected by fibrotic diseases are kidney (Klein J. et al. (2011)) and lung (Coward W R et al. (2010)). In these organs, administration of Relaxin exhibits a strong anti-fibrotic activity (Bennett R G (2009)). Therefore, protease cleavage sites as linker are preferred from proteases mainly expressed in kidney and/or lung, such as MMP12 in the lung (Garbacki N. et al. (2009)) or Renin in the kidney (Castrop H. et al. (2010)).

The protease cleavage site of endo-proteases are known in the art. Some examples are given in table 1.

TABLE 1
Examples for Proteases and their corresponding cleavage sites.

| | P4 | P3 | P2 | P1 | P1′ | P2′ | P3′ | P4′ | |
|---|---|---|---|---|---|---|---|---|---|
| coagulation factor VIIa | Lys | Leu | Thr | Arg | Ala | Glu | Thr | Val | Morrissey, 2004 |
| | Asp | Phe | Thr | Arg | Val | Val | Gly | Gly | Morrissey, 2004 |
| | Met | Ala | Thr | Arg | Lys | Met | His | Asp | Safa et al., 1999 |
| | Leu | Ile | Gln | Arg | Asn | Leu | Ser | Pro | Safa et al., 1999 |
| cathepsin S | Cys | Pro | Val | Thr | Tyr | Gly | Gln | Cys | Taggart et al., 2001 |
| | Gln | Ala | Ser | Arg | Ser | Phe | Asn | Gln | Cirman et al., 2004 |
| | Ser | Gly | Leu | Gly | Ala | Glu | His | Ile | Cirman et al., 2004 |
| | Val | Gln | Ala | Tyr | Trp | Glu | Ala | Asp | Cirman et al., 2004 |
| coagulation factor Xa | Lys | Arg | Gly | Arg | Lys | Gln | Cys | Lys | Haas et al., 1997 |
| | Ala | Thr | Glu | Arg | Thr | Thr | Ser | Ile | Haas et al., 1997 |
| | Ser | Glu | Pro | Arg | Ile | Ser | Tyr | Gly | Haas et al., 1997 |
| | Ala | Ala | Asp | Arg | Gly | Leu | Thr | Thr | Haas et al., 1997 |
| | Ala | Glu | Phe | Arg | His | Asp | Ser | Gly | Haas et al., 1997 |
| ADAMTS1 | Ile | Pro | Glu | Asn | Phe | Phe | Gly | Val | Rodgriguez-Manzaneque et al., 2002 |
| | Lys | Glu | Glu | Glu | Gly | Leu | Gly | Ser | Rodgriguez-Manzaneque et al., 2002 |
| | Thr | Glu | Gly | Glu | Ala | Arg | Gly | Ser | Rodgriguez-Manzaneque et al., 2002 |
| | Ser | Glu | Leu | Glu | Gly | Arg | Gly | Thr | Rodgriguez-Manzaneque et al., 2002 |
| ADAM12 | Leu | Ala | Gln | Ala | HPh | Arg | Ser | LyN | Moss & Rasmussen, 2007 |
| complement | Ser | Val | Ala | Arg | Thr | Leu | Leu | Val | Duncan et al., 2008 |
| component activated | Ser | Leu | Gly | Arg | Lys | Ile | Gln | Ile | Arlaud et al., 2004 |
| C1s | Gly | Leu | Gln | Arg | Ala | Leu | Glu | Ile | Sim & Tsiftsoglou, 2004 |
| napsin A | Lys | Leu | Val | Leu | Pro | Val | Leu | Pro | Ueno et al., 2004 |
| renin | Pro | Phe | His | Leu | Leu | Val | His | Ser | Suzuki et al., 2004 |
| | Pro | Phe | His | Leu | Val | Ile | His | Asn | Suzuki et al., 2004 |
| | Pro | Tyr | Ile | Leu | Lys | Arg | Gly | Ser | Dunn, 2004 |
| elastase-1 | Gly | Leu | Arg | Val | Gly | Phe | Tyr | Glu | Mortensen et al., 1981 |
| | Leu | Arg | Val | Gly | Phe | Tyr | Glu | Ser | Mortensen et al., 1981 |
| | Pro | Asn | Val | Ile | Leu | Ala | Pro | Ser | Edelstein et al., 1997 |
| MMP2 | Tyr | Arg | Ile | Ile | Gly | Tyr | Thr | Pro | auf dem Keller et al., 2010 |
| | Arg | Phe | Ser | Arg | Ile | His | Asp | Gly | auf dem Keller et al., 2010 |
| | Pro | Glu | Ile | Cys | Lys | Gln | Asp | Ile | auf dem Keller et al., 2010 |
| | Phe | Leu | Gly | Asn | Lys | Tyr | Glu | Ser | auf dem Keller et al., 2010 |
| MMP9 | Arg | Ala | Lys | Arg | Phe | Ala | Ser | Leu | Tortorella et al., 2005 |
| | Ile | Pro | Glu | Asn | Phe | Phe | Gly | Val | Fosang et al., 1992 |
| | Ile | Pro | Glu | Asn | Phe | Phe | Gly | Val | Fosang et al., 1992 |
| | Pro | Phe | Phe | Pro | Phe | His | Ser | Pro | Starckx et al., 2003 |
| urokinase | Arg | Gly | Ser | Val | Ile | Leu | Thr | Val | Fosang et al., 1998 |
| | Pro | Ser | Ser | Arg | Arg | Arg | Val | Asn | Pawar et al., 2007 |
| | Cys | Pro | Gly | Arg | Val | Val | Gly | Gly | Robbins et al., 1967 |
| | Ser | Ser | Ser | Arg | Gly | Pro | Tyr | His | Vakili et al., 2001 |
| Chymase | Arg | Val | Gly | Phe | Tyr | Glu | Ser | Asp | Walter et al., 1999 |
| | Val | Gly | Phe | Tyr | Glu | Ser | Asp | Val | Walter et al., 1999 |
| | Ile | His | Pro | Phe | His | Leu | — | — | Caughey et al., 2000 |
| | Asp | Arg | Val | Tyr | Ile | His | Pro | Phe | Raymond et al., 2003 |
| Thrombin | Ala | Ala | Pro | Arg | Ala | Gly | Leu | Ala | Lam et al., 2007 |
| | Pro | Gln | Pro | Arg | Arg | Leu | Leu | Pro | Lam et al., 2007 |
| | Phe | Gly | Leu | Arg | Phe | Tyr | Ala | Tyr | Ireland et al., 1998 |
| | Ile | Ala | Gly | Arg | Ser | Leu | Asn | Pro | Björk et al., 1981 |
| Trypsin | Ile | Asn | Ala | Arg | Val | Ser | Thr | Ile | Szmola & Sahin-Toth, 2007 |
| | Gln | Lys | Ser | Arg | Asn | Gly | Leu | Arg | Rossmann et al., 2002 |
| | Pro | Arg | Thr | Arg | Asn | Ala | Met | Arg | Johnson & Bond, 1997 |
| | Gly | Cys | Thr | Lys | Ile | Tyr | Asp | Pro | Witt et al., 2000 |

It is well-known in the art that variations of protease cleavage sites may lead to different turn-over of the substrates. Such variations include conservative or non-conservative exchange of one or more amino acids within the recognition sequence and can influence the kcat and/or Km of the turnover of the substrate. Thus, varying the PCS in the Relaxin fusion protein provides a basis to further tailor the release kinetics of Relaxin.

As the preferred cleavage sites of endoproteases are known, a PCS/endoprotease combination is selected so that the endoprotease specifically cleaves the PCS but does not cleave Relaxin or the half-life extending moiety. Furthermore, there are methods provided in the art to determine whether an endo-protease also hydrolyzes peptide bonds of the Relaxin or the half-life extending moiety.

A preferred PCS is a cleavage site of coagulation factor Xa, further preferred is a PCS having the sequence IleGluGlyArgMetAsp.

In a further embodiment the PCS linker polypeptide of the aforementioned fusion polypeptides/proteins may further have a stretcher polypeptide at the N-terminus and/or at the C-terminus. A stretcher unit may provide better access of an endo-protease to the PCS, hence provide better release of Relaxin from the fusion protein. Methods to determine a protease activity on a given substrate are known in the art. Such stretchers are known in the art and are 1 to about 100 amino acids in length, are 1 to about 50 amino acids in length, are 1 to about 25 amino acids in length, are 1 to about 15 amino acids in length, are 1 to 10 amino acids in length, or are 1 to 5 amino acids in length.

The amino acid composition of stretcher sequences is variable, although a stretcher exhibiting a low immunogenicity potential is preferred. In an embodiment of the invention a stretcher polypeptide can be composed of any amino acid. In a more preferred embodiment the stretcher polypeptide comprises Gly and Ser residues. In a further preferred embodiment the stretcher peptide is a glycine-rich linker such as peptides comprising the sequence [GGGGS]ₙ as disclosed in U.S. Pat. No. 7,271,149, n being an integer number between 1 and 20, preferably between 1 and 10, more preferably between 1 and 5 and even more preferably between 1 and 3. In other embodiments, a serine-rich strecher polypeptide is used, as described in U.S. Pat. No. 5,525,491. A further preferred embodiment is a stretcher polypeptide which comprises Gly and Ser residues and has a ratio of Gly to Ser of at least 3 to 1.

When a stretcher unit is introduced between the PCS and the Relaxin the stretcher unit will remain on the Relaxin after cleavage by the respective endo-protease, in addition to the P or P′ amino acids of the PCS, respectively. Therefore, stretcher units are used which will not diminish Relaxin activity. In a preferred embodiment the stretcher unit is inserted between the PCS and the half-life extending moiety.

In a further embodiment the aforementioned fusion polypeptides release active Relaxin. In a further preferred embodiment the Relaxin activity is activation of the relaxin receptor LGR7. Methods for determining Relaxin activity are known in the art or are provided herein. In an even further preferred embodiment, the activation of the relaxin receptor LGR7 is determined by a method disclosed in experimental methods herein. In an even further preferred embodiment, the determination of the activation of the Relaxin receptor LGR7 is determining an EC50 value. In an even more preferred embodiment the aforementioned Relaxin activity is less than 10⁵ fold, 10⁴ fold, 10³ fold, 100 fold, 75 fold, 50 fold, 25 fold or 10 fold lower compared to the corresponding wild type Relaxin effective concentration inducing a half maximal activity. For example, the corresponding wild type Relaxin for a fusion polypeptide based on human Relaxin 2 is the human Relaxin 2 protein.

### Half-Life Extension Via Proteinaceous Half-Life Extending Moieties:

To improve the half-life of a fusion polypeptide of the invention a fusion with a proteinaceous half-life extending moiety is contemplated, such as the immunoglobulin Fc fragment of immunoglobulins, transferrin, transferrin receptor or at least the transferrin-binding portion thereof, serum albumin, or variants thereof or binding modules that bind in-vivo to other molecules mediating longer half-life, e.g. serum albumin binding protein.

“Immunoglobulins” are molecules containing polypeptide chains held together by disulfide bonds, typically having two light chains and two heavy chains. In each chain, one domain (variable domain Fv) has a variable amino acid sequence depending on the antibody specificity of the molecule. The other domains (constant domains C) have a rather constant sequence common to molecules of the same class.

As used herein, the “Fc” portion of an immunoglobulin has the meaning commonly given to the term in the field of immunology. Specifically, this term refers to an antibody fragment that is obtained by removing the two antigen binding regions (the Fab fragments) from the antibody. One way to remove the Fab fragments is to digest the immunoglobulin with papain protease. Thus, the Fc portion is formed from approximately equally sized fragments of the constant region from both heavy chains, which associate through non-covalent interactions and optionally disulfide bonds. The Fc portion can include the hinge regions and extend through the CH₂ and CH3 domains to the C-terminus of the antibody. Representative hinge regions for human and mouse immunoglobulins can be found in Antibody Engineering, A Practical Guide, Borrebaeck, C. A. K., ed., W.H. Freeman and Co., 1992.

There are five types of human immunoglobulin Fc regions with different effector and pharmacokinetic properties: IgG, IgA, IgM, IgD, and IgE. IgG is the most abundant immunoglobulin in serum. IgG also has the longest half-life in serum of any immunoglobulin (23 days). Unlike other immunoglobulins. IgG is efficiently recirculated after endocytosis following binding to an Fc receptor. There are four IgG subclasses G1, G2, G3, and G4, each of which has different effect or functions. These effector functions are generally mediated through interaction with the Fc receptor (FcγR) or by binding C1q and fixing complement. Binding to FcγR can lead to antibody dependent cell mediated cytolysis, whereas binding to complement factors can lead to complement mediated cell lysis. In designing heterologous Fc fusion proteins wherein the Fc portion is being utilized solely for its ability to extend half-life, it is important to minimize any effector function. All IgG subclasses are capable of binding to Fc receptors (CD16, CD32, CD64) with G1 and G3 being more effective than G2 and G4. The Fc receptor binding region of IgG is formed by residues located in both the hinge and the carboxy terminal regions of the CH2 domain.

Depending on the desired in vivo effect, the heterologous fusion proteins of the present invention may contain any of the isotypes described above or may contain mutated Fc regions wherein the complement and/or Fe receptor binding functions have been altered. Thus, the heterologous fusion proteins of the present invention may contain the entire Fc portion of an immunoglobulin, fragments of the Fc portion of an immunoglobulin, or analogs thereof.

It is preferable that the Fc region used for the heterologous fusion proteins of the present invention be derived from an IgG1 or an IgG2 Fc region.

Generally, the Fc region used for the heterologous fusion proteins of the present invention can be derived from any species including but not limited to human, rat, mouse and pig. Preferably, the Fc region used for the present invention is derived from human or rat. However, most preferred are human Fc regions and fragments and variants thereof to reduce the risk of the fusion protein being immunogenic in humans. A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification. Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95% sequence identity therewith.

The Relaxin compounds described above can be fused directly or via a peptide stretcher to albumin or an analog, fragment, or derivative thereof. Generally the albumin proteins making up part of the fusion proteins of the present invention can be derived from albumin cloned from any species. However, human albumin and fragments and analogs thereof are preferred to reduce the risk of the fusion protein being immunogenic in humans. Human serum albumin (HSA) consists of a single non-glycosylated polypeptide chain of 585 amino acids with a formula molecular weight of 66,500. The amino acid sequence of HSA (SEQ ID NO: 3) has been described e.g. in Meloun, et al. (1975); Behrens, et al. (1975); Lawn, et al. (1981) and Minghetti, et al. (1986). A variety of polymorphic variants as well as analogs and fragments of albumin have been described (see Weitkamp, et al. (1973)). For example, in EP0322094 and EP0399666 various fragments of human serum albumin are disclosed. It is understood that the heterologous fusion proteins of the present invention include Relaxin compounds comprising any albumin protein including fragments, analogs, and derivatives wherein such fusion protein is biologically active and has a longer plasma half-life than the corresponding wild type Relaxin alone. Thus, the albumin portion of the fusion protein need not necessarily have a plasma half-life equal to that of native human albumin. Fragments, analogs, and derivatives are known or can be generated that have longer half-lives or have half-lives intermediate to that of native human albumin and the Relaxin compound of interest. The techniques are well-known in the art, see, e.g., WO 93/15199, WO 93/15200, WO 01/77137 and EP0413622.

In an embodiment of the invention the proteinaceous half-life extending moiety has low immunogenicity, is human or humanized. In a preferred embodiment the proteinaceous half-life extending moiety is human, such as human transferrin (SEQ ID NO: 2), human serum albumin (SEQ ID NO: 3), or human IgG1 Fc (SEQ ID NO: 4).

Additionally, other proteins, protein domains or peptides improving the biological half life can also be used as fusion partners.

Half-life extension via fusion to human serum albumin is disclosed for example in WO93/15199. Albumin binding as a general strategy for improving the pharmacokinetics of proteins is described for example in Dennis et al., The Journal of Biological Chemistry, Vol. 277, No 38, Issue of September 20, pp. 35035-35043. Half-life extension via fusion to human serum albumin binding proteins is disclosed for example in US20100104588. Half-life extension via fusion to human serum albumin or IgG-Fc binding proteins is disclosed for example in WO01/45746. A further example of half-life extension via fusion to human serum albumin binding peptides is disclosed in WO2010/054699.

Half-life extension via fusion to an Fc domain is disclosed for example in WO2001/058957.

The biological activity determines the preferred orientation of the protein of interest to its fusion partner. C-terminal as well as N-terminal orientations of fusion partners are included. In addition, for improvement of the biological half life or other functions, fusion partners may be modified by phosphorylation, sulfation, acrylation, glycosylation, deglycosylation, methylation, farnesylation, acetylation, amidation or others.

Examples of proteinaceous half-life extending moieties are transferrin, transferrin receptor or at least the transferrin-binding portion thereof, serum albumin, serum albumin binding proteins. Immunglobulins, and the Fc domain of an immunoglobulin.

Preferred are human proteinaceous half-life extending moieties, e.g human transferrin, human transferrin receptor or at least the transferrin-binding portion thereof, human serum albumin, human immunoglobulin or human Fe domains.

In a further embodiment the aforementioned fusion polypeptides comprising at least one half-life extending moiety have an extended half-life compared to the corresponding wild type Relaxin, wherein the half-life extension is at least 5, 10, 20, 50, 100 or 500-fold. Preferably, the half-life is determined as serum half-life, meaning detection of the fusion protein in serum or whole blood, for example by using a commercially available quantification ELISA assay (e.g. R&D Systems, Human Relaxin-2 Quantikine ELISA kit, catalogue number DRL200). The half-life is preferably a human blood half-life.

### Cloning, Vector Systems, Expression, Hosts, and Purification

The invention also provides a vector which comprises an isolated nucleic acid molecule encoding a fusion polypeptide HEM-PCS-proRelaxin or proRelaxin-PCS-HEM of the invention. This vector system is operatively linked to an expression sequence capable of directing its expression in a host cell.

A suitable host cell may be selected from the group consisting of bacterial cells (such as E. coli), yeast cells (such as Saccharomyces cerevisiae), fungal cells, plant cells, insect cells and animals cells. Animal cells include, but are not limited to. HEK293 cells, CHO cells, COS cells, BHK cells, HeLa cells and various primary mammalian cells. Derivatives of mammalian cells such as HEK293T cells are also applicable.

### DNA Molecules of the Invention

The present invention also relates to the DNA molecules that encode a fusion protein HEM-PCS-proRelaxin or proRelaxin-PCS-HEM of the invention.

DNA molecules of the invention are not limited to the sequences disclosed herein, but also include variants thereof. DNA variants within the invention may be described by reference to their physical properties in hybridization. The skilled worker will recognize that DNA can be used to identify its complement and, since DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among DNA sequences based on their structural relatedness to a particular probe. For guidance regarding such conditions see. Sambrook et al., 1989 supra and Ausubel et al., 1995 (Ausubel, F. M., Brent, R., Kingston, R. E., Moore, D. D., Sedman, J. G., Smith, J. A., & Struhl, K. eds. (1995). Current Protocols in Molecular Biology. New York: John Wiley and Sons). Structural similarity between two polynucleotide sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences. The following relationships are useful in correlating hybridization and relatedness (where Tₘ is the melting temperature of a nucleic acid duplex):
a. Tₘ=69.3+0.41(G+C) %
b. The Tₘ of a duplex DNA decreases by 1° C. with every increase of 1% in the number of mismatched base pairs.
c. (Tₘ)_{μ2}−(Tₘ)_{μ1}/=18.5 log₁₀ μ2/μ1
   where μl and μ2 are the ionic strengths of two solutions.

Hybridization stringency is a function of many factors, including overall DNA concentration, ionic strength, temperature, probe size and the presence of agents which disrupt hydrogen bonding. Factors promoting hybridization include high DNA concentrations, high ionic strengths, low temperatures, longer probe size and the absence of agents that disrupt hydrogen bonding. Hybridization typically is performed in two phases: the “binding” phase and the “washing” phase.

First, in the binding phase, the probe is bound to the target under conditions favoring hybridization. Stringency is usually controlled at this stage by altering the temperature. For high stringency, the temperature is usually between 65° C. and 70° C., unless short (<20 nt) oligonucleotide probes are used. A representative hybridization solution comprises 6×SSC, 0.5% SDS, 5×Denhardt's solution and 100 μg of non-specific carrier DNA. See Ausubel et al., section 2.9, supplement 27 (1994). Of course, many different, yet functionally equivalent, buffer conditions are known.

Where the degree of relatedness is lower, a lower temperature may be chosen. Low stringency binding temperatures are between about 25° C. and 40° C. Medium stringency is between at least about 40° C. to less than about 65° C. High stringency is at least about 65° C.

Second, the excess probe is removed by washing. It is at this phase that more stringent conditions usually are applied. Hence, it is this “washing” stage that is most important in determining relatedness via hybridization. Washing solutions typically contain lower salt concentrations. One exemplary medium stringency solution contains 2×SSC and 0.1% SDS. A high stringency wash solution contains the equivalent (in ionic strength) of less than about 0.2×SSC, with a preferred stringent solution containing about O.1×SSC. The temperatures associated with various stringencies are the same as discussed above for “binding.” The washing solution also typically is replaced a number of times during washing. For example, typical high stringency washing conditions comprise washing twice for 30 minutes at 55° C. and three times for 15 minutes at 60° C.

An embodiment of the invention is an isolated nucleic acid sequence that encodes a fusion polypeptide of the invention.

### Recombinant DNA Constructs and Expression

The present invention further provides recombinant DNA constructs comprising one or more of the nucleotide sequences of the present invention. The recombinant constructs of the present invention are used in connection with a vector, such as a plasmid, phagemid, phage or viral vector, into which a DNA molecule encoding a fusion polypeptide of the invention is inserted.

A fusion polypeptide as provided herein can be prepared by recombinant expression of nucleic acid sequences encoding a fusion polypeptide in a host cell. To express a fusion polypeptide recombinantly, a host cell can be transfected with a recombinant expression vectors carrying DNA fragments encoding a fusion polypeptide such that the fusion polypeptide is expressed in the host cell. Standard recombinant DNA methodologies are used to prepare and/or obtain nucleic acids encoding a fusion polypeptide, incorporate these nucleic acids into recombinant expression vectors and introduce the vectors into host cells, such as those described in Sambrook. Fritsch and Maniatis (eds.), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

To express the fusion polypeptide standard recombinant DNA expression methods can be used (see, for example, Goeddel; Gene Expression Technology. Methods in Enzymology 185, Academic Press, San Diego, Calif. (1990)). For example, DNA encoding the desired polypeptide can be inserted into an expression vector which is then transfected into a suitable host cell. Suitable host cells are prokaryotic and eukaryotic cells. Examples for prokaryotic host cells are e.g. bacteria, examples for eukaryotic host cells are yeast, insect or mammalian cells. It is understood that the design of the expression vector, including the selection of regulatory sequences is affected by factors such as the choice of the host cell, the level of expression of protein desired and whether expression is constitutive or inducible.

### Bacterial Expression

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus.

Bacterial vectors may be, for example, bacteriophage-, plasmid- or phagemid-based. These vectors can contain a selectable marker and bacterial origin of replication derived from commercially available plasmids typically containing elements of the well known cloning vector pBR322 (ATCC 37017). Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

In bacterial systems, a number of expression vectors may be advantageously selected depending upon the use intended for the protein being expressed. For example, when a large quantity of such a protein is to be produced vectors which direct the expression of high levels of fusion polypeptide products that are readily purified may be desirable. Fusion polypeptide of the present invention include purified products, products of chemical synthetic procedures, and products produced by recombinant techniques from a prokaryotic host, including, for example, E. coli, Bacillus subtilis, Salmonella typhimurium and various species within the genera Pseudomonas, Streptomyces, and Staphylococcus, preferably, from E. coli cells.

### Mammalian Expression and Purification

Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al. The recombinant expression vectors can also include origins of replication and selectable markers (see e.g., U.S. Pat. Nos. 4,399,216, 4,634,665 and U.S. Pat. No. 5,179,017, by Axel et al.). Suitable selectable markers include genes that confer resistance to drugs such as G418, hygromycin or methotrexate, on a host cell into which the vector has been introduced. For example, the dihydrofolate reductase (DHFR) gene confers resistance to methotrexate and the neo gene confers resistance to G418. Transfection of the expression vector into a host cell can be carried out using standard techniques such as electroporation, calcium-phosphate precipitation, and DEAE-dextran, lipofection or polycation-mediated transfection.

Suitable mammalian host cells for expressing the fusion polypeptides provided herein include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621, NSO myeloma cells. COS cells and SP2 cells. In some embodiments, the expression vector is designed such that the expressed protein is secreted into the culture medium in which the host cells are grown. Transient transfection/epression of antibodies can for example be achieved following the protocols by Durocher et al (2002) Nucl. Acids Res. Vol 30 e9. Stable transfection/expression of antibodies can for example be achieved following the protocols of the UCOE system (T. Benton et al. (2002) Cytotechnology 38: 43-46). The fusion polypeptide can be recovered from the culture medium using standard protein purification methods.

A fusion polypeptide of the invention can be recovered and purified from recombinant cell cultures by well-known methods including, but not limited to ammonium sulfate or ethanol precipitation, acid extraction, Protein A chromatography, Protein G chromatography, anion or cation exchange chromatography, phospho-cellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Fusion polypeptides of the invention include purified or isolated products, products of chemical synthetic procedures, and products produced by recombinant techniques from a eukaryotic host, including, for example, yeast (for example Pichia), higher plant, insect and mammalian cells, preferably from mammalian cells. Depending upon the host employed in a recombinant production procedure, the fusion polypeptide of the present invention can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20.

### Therapeutic Use

An embodiment of the invention is the use of a pharmaceutical composition or a fusion polypeptide of the invention in the treatment of cardiovascular diseases, kidney diseases, pancreatitis, inflammation, cancer, scleroderma, pulmonary, renal, and hepatic fibrosis.

### Cardiovascular Diseases

Disorders of the cardiovascular system, or cardiovascular disorders, mean in the context of the present invention for example the following disorders: hypertension (high blood pressure), peripheral and cardiac vascular disorders, coronary heart disease, stable and unstable angina pectoris, myocardial insufficiency, persistent ischemic dysfunction (“hibernating myocardium”), temporary postischemic dysfunction (“stunned myocardium”), heart failure, disturbances of peripheral blood flow, acute coronary syndrome, heart failure and myocardial infarction.

In the context of the present invention, the term heart failure includes both acute and chronic manifestations of heart failure, as well as more specific or related types of disease, such as acute decompensated heart failure, right heart failure, left heart failure, global failure, ischemic cardiomyopathy, dilated cardiomyopathy, congenital heart defects, heart valve defects, heart failure associated with heart valve defects, mitral stenosis, mitral insufficiency, aortic stenosis, aortic insufficiency, tricuspid stenosis, tricuspid insufficiency, pulmonary stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, and diastolic and systolic heart failure and acute phases of worsening heart failure.

The compounds according to the invention are further also suitable for reducing the area of myocardium affected by an infarction, and for the prophylaxis of secondary infarctions.

The compounds according to the invention are furthermore suitable for the prophylaxis and/or treatment of thromboembolic disorders, reperfusion damage following ischemia, micro- and macrovascular lesions (vasculitis), arterial and venous thromboses, edemas, ischemias such as myocardial infarction, stroke and transient ischemic attacks, for cardio protection in connection with coronary artery bypass operations (CABG), primary percutaneous transluminal coronary angioplasties (PTCAs). PTCAs after thrombolysis, rescue PTCA, heart transplants and open-heart operations, and for organ protection in connection with transplants, bypass operations, catheter examinations and other surgical procedures.

Other areas of indication are, for example, the prevention and/or treatment of respiratory disorders, such as, for example, chronic obstructive pulmonary disease (chronic bronchitis, COPD), asthma, pulmonary emphysema, bronchiectases, cystic fibrosis (mucoviscidosis) and pulmonary hypertension, in particular pulmonary arterial hypertension.

### Kidney Disease

The present invention relates to the use of a fusion polypeptide of the invention as a medicament for the prophylaxis and/or treatment of kidney diseases, especially of acute and chronic kidney diseases and acute and chronic renal insufficiencies, as well as acute and chronic renal failure, including acute and chronic stages of renal failure with and without the requirement of dialysis, as well as the underlying or related kidney diseases such as renal hypoperfusion, dialysis induced hypotension, glomerulopathies, glomerular and tubular proteinuria, renal edema, hematuria, primary, secondary, as well as acute and chronic glomerulonephritis, membranous and membranoproliferative glomerulonephritis. Alport-Syndrome, glomerulosclerosis, interstistial tubular diseases, nephropathic diseases, such as primary and inborn kidney diseases, renal inflammation, immunological renal diseases like renal transplant rejection, immune complex induced renal diseases, as well as intoxication induced nephropathic diseases, diabetic and non-diabetic renal diseases, pyelonephritis, cystic kidneys, nephrosclerosis, hypertensive nephrosclerosis, nephrotic syndrome, that are characterized and diagnostically associated with an abnormal reduction in creatinine clearance and/or water excretion, abnormal increased blood concentrations of urea, nitrogen, potassium and/or creatinine, alteration in the activity of renal enzymes, such as glutamylsynthetase, urine osmolarity and urine volume, increased microalbuminuria, macroalbuminuria, glomerular and arteriolar lesions, tubular dilation, hyperphosphatemia and/or the requirement of dialysis.

In addition, a fusion polypeptide of the invention can be used as a medicament for the prophylaxis and/or treatment of renal carcinomas, after incomplete resection of the kidney, dehydration after overuse of diuretics, uncontrolled blood pressure increase with malignant hypertension, urinary tract obstruction and infection, amyloidosis, as well as systemic diseases associated with glomerular damage, such as Lupus erythematodes, and rheumatic immunological systemic diseases, as well as renal artery stenosis, renal artery thrombosis, renal vein thrombosis, analgetics induced nephropathy and renal tubular acidosis.

In addition, a fusion polypeptide of the invention can be used as a medicament for the prophylaxis and/or treatment of contrast medium induced and drug induced acute and chronic interstitial kidney diseases, metabolic syndrome and dyslipemia.

In addition, the present invention includes the use of a fusion polypeptide of the invention as a medicament for the prophylaxis and/or treatment of aftereffects associated with acute and/or chronic kidney diseases, such as pulmonary edema, heart failure, uremia, anemia, electrolyte disturbances (e.g. hyperkalemia, hyponatremia), as well as bony and carbohydrate metabolism.

### Lung Diseases

Furthermore, the fusion proteins according to the invention are also suitable for the treatment and/or prophylaxis of lung diseases especially of asthmatic disorders, pulmonary arterial hypertension (PAH) and other forms of pulmonary hypertension (PH) including left-heart disease, HIV, sickle cell anaemia, thromboembolisms (CTEPH), sarkoidosis, COPD or pulmonary fibrosis-associated pulmonary hypertension, chronic-obstructive pulmonary disease (COPD), acute respiratory distress syndrome (ARDS), acute lung injury (ALI), alpha-1-antitrypsin deficiency (AATD), pulmonary fibrosis, pulmonary emphysema (for example pulmonary emphysema induced by cigarette smoke) and cystic fibrosis (CF).

### Fibrotic Disorders

The fusion proteins according to the invention are furthermore suitable for the treatment and/or prophylaxis of fibrotic disorders of the internal organs such as, for example, the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitreoretinopathy and disorders of the connective tissue (for example sarcoidosis).

### Cancer

Cancer is disease in which a group of cells display uncontrolled growth. Cancers are usually classified in carcinomas which is a cancer derived from epithelial cells (This group includes many of the most common cancers, including those of the breast, prostate, lung and colon.); sarcomas, which are derived from connective tissue, or mesenchymal cells; lymphoma and leukemia, derived from hematopoietic cells; germ cell tumor, which is derived from pluripotent; and blastomas, which is a cancer derived from immature “precursor” or embryonic tissue.

The present invention furthermore provides the use of a fusion protein of the invention for preparing a medicament for the treatment and/or prevention of disorders, in particular the disorders mentioned above.

The present invention furthermore provides a method for the treatment and/or prevention of disorders, in particular the disorders mentioned above, using an effective amount of at least one fusion proteins of the invention.

The present invention furthermore provides a fusion proteins of the invention for use in a method for the treatment and/or prophylaxis of coronary heart disease, acute coronary syndrome, heart failure, and myocardial infarction.

### Pharmaceutical Compositions and Administration

The present invention also provides for pharmaceutical compositions comprising a Relaxin fusion protein in a pharmacologically acceptable vehicle. The Relaxin fusion protein may be administrated systemically or locally. Any appropriate mode of administration known in the art may be used including, but not limited to, intravenous, intraperitoneal, intraarterial, intranasal, by inhalation, oral, subcutaneous administration, by local injection or in form of a surgical implant.

The present invention also relates to pharmaceutical compositions which may comprise inventive fusion polypeptides, alone or in combination with at least one other agent, such as stabilizing compound, which may be administered in any sterile, biocompatible pharmaceutical carrier, including, but not limited to, saline, buffered saline, dextrose, and water. Any of these molecules can be administered to a patient alone, or in combination with other agents, drugs or hormones, in pharmaceutical compositions where it is mixed with excipient(s) or pharmaceutically acceptable carriers. In one embodiment of the present invention, the pharmaceutically acceptable carrier is pharmaceutically inert.

The present invention also relates to the administration of pharmaceutical compositions. Such administration is accomplished orally. or parenterally. Methods of parenteral delivery include topical, intra-arterial, intramuscular, subcutaneous, intramedullary, intrathecal, intraventricular, intravenous, intraperitoneal, or intranasal administration. In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer's solution, or physiologically buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

A fusion protein according to the invention can be used alone or, if required, in combination with other active compounds. The present invention furthermore provides medicaments comprising at least one fusion polypeptide according to the invention and one or more further active ingredients, in particular for the treatment and/or prevention of the disorders mentioned above.

Suitable active ingredients for combination are, by way of example and by way of preference: active ingredients which modulate lipid metabolism, anti-diabetics, hypotensive agents, perfusion-enhancing and/or antithrombotic agents, antioxidants, chemokine receptor antagonists, p38-kinase inhibitors. NPY agonists, orexin agonists, anorectics. PAF-AH inhibitors, anti-phlogistics (COX inhibitors, LTB4-receptor antagonists), analgesics for example aspirin, antidepressants and other psychopharmaceuticals.

The present invention relates in particular to combinations of at least one of the fusion polypeptides according to the invention with at least one lipid metabolism-altering active ingredient, anti-diabetic, blood pressure reducing active ingredient and/or agent having antithrombotic effects.

The fusion polypeptides according to the invention can preferably be combined with one or more
lipid metabolism-modulating active ingredients, by way of example and by way of preference from the group of the HMG-CoA reductase inhibitors, inhibitors of HMG-CoA reductase expression, squalene synthesis inhibitors. ACAT inhibitors, LDL receptor inductors, cholesterol absorption inhibitors, polymeric bile acid adsorbers, bile acid reabsorption inhibitors. MTP inhibitors, lipase inhibitors. LpL activators, fibrates, niacin. CETP inhibitors, PPAR-α, PPAR-γ and/or PPAR-δ agonists, RXR modulators, FXR modulators, LXR modulators, thyroid hormones and/or thyroid mimetics. ATP citrate lyase inhibitors. Lp(a) antagonists, cannabinoid receptor 1 antagonists, leptin receptor agonists, bombesin receptor agonists, histamine receptor agonists and the antioxidants/radical scavengers;
antidiabetics mentioned in the Rote Liste 2004/II, chapter 12, and also, by way of example and by way of preference, those from the group of the sulfonylureas, biguanides, meglitinide derivatives, glucosidase inhibitors, inhibitors of dipeptidyl-peptidase IV (DPP-IV inhibitors), oxadiazolidinones, thiazolidinediones. GLP 1 receptor agonists, glucagon antagonists, insulin sensitizers. CCK 1 receptor agonists, leptin receptor agonists, inhibitors of liver enzymes involved in the stimulation of gluconeogenesis and/or glycogenolysis, modulators of glucose uptake and also potassium channel openers, such as, for example, those disclosed in WO 97/26265 and WO 99/03861;
hypotensive active ingredients, by way of example and by way of preference from the group of the calcium antagonists, angiotensin All antagonists, ACE inhibitors, renin inhibitors, beta-receptor blockers, alpha-receptor blockers, aldosterone antagonists, mineralocorticoid receptor antagonists, ECE inhibitors, ACE/NEP inhibitors and the vasopeptidase inhibitors; and/or
antithrombotic agents, by way of example and by way of preference from the group of the platelet aggregation inhibitors or the anticoagulants;
diuretics;
vasopressin receptor antagonists;
organic nitrates and NO donors;
compounds with positive inotropic activity;
compounds which inhibit the degradation of cyclic guanosine monophosphate (cGMP) and/or cyclic adenosine monophosphat (cAMP), such as, for example, inhibitors of phosphodiesterases (PDE) 1, 2, 3, 4 and/or 5, in particular PDE 5 inhibitors, such as sildenafil, vardenafil and tadalafil, and also PDE 3 inhibitors, such as milrinone;
natriuretic peptides, such as, for example, “atrial natriuretic peptide” (ANP, anaritide), “B-type natriuretic peptide” or “brain natriuretic peptide” (BNP, nesiritide), “C-type natriuretic peptide” (CNP) and also urodilatin;
agonists of the prostacyclin receptor (IP receptor), such as, by way of example, iloprost, beraprost, cicaprost;
inhibitors of the I_{f} (funny channel) channel, such as, by way of example, ivabradine;
calcium sensitizers, such as, by way of example and by way of preference, levosimendan;
potassium supplements;
NO-independent, but heme-dependent stimulators of guanylate cyclase, such as, in particular, the compounds described in WO 00/06568, WO 00/06569, WO 02/42301 and WO 03/095451;
NO- and heme-independent activators of guanylate cyclase, such as, in particular, the compounds described in WO 01/19355, WO 01/19776, WO 01/19778, WO 01/19780, WO 02/070462 and WO 02/070510;
inhibitors of human neutrophil elastase (HNE), such as, for example, sivelestat and DX-890 (Reltran);
compounds which inhibit the signal transduction cascade, such as, for example, tyrosine-kinase inhibitors, in particular sorafenib, imatinib, gefitinib and erlotinib; and/or
compounds which modulate the energy metabolism of the heart, such as, for example, etomoxir, dichloroacetate, ranolazine and trimetazidine.

Lipid metabolism-modifying active ingredients are to be understood as meaning, preferably, compounds from the group of the HMG-CoA reductase inhibitors, squalene synthesis inhibitors. ACAT inhibitors, cholesterol absorption inhibitors. MTP inhibitors, lipase inhibitors, thyroid hormones and/or thyroid mimetics, niacin receptor agonists. CETP inhibitors, PPAR-α agonists PPAR-γ agonists, PPAR-δ agonists, polymeric bile acid adsorbers, bile acid reabsorption inhibitors, antioxidants/radical scavengers and also the cannabinoid receptor 1 antagonists.

In a preferred embodiment of the invention, a fusion polypeptide according to the invention is administered in combination with an HMG-CoA reductase inhibitor from the class of the statins, such as, by way of example and by way of preference, lovastatin, simvastatin, pravastatin, fluvastatin, atorvastatin, rosuvastatin or pitavastatin.

In a preferred embodiment of the invention, the fusion polypeptides according to the invention are administered in combination with a squalene synthesis inhibitor, such as, by way of example and by way of preference, BMS-188494 or TAK-475.

In a preferred embodiment of the invention, the fusion polypeptides according to the invention are administered in combination with an ACAT inhibitor, such as, by way of example and by way of preference, avasimibe, melinamide, pactimibe, eflucimibe or SMP-797.

In a preferred embodiment of the invention, the fusion proteins according to the invention are administered in combination with a cholesterol absorption inhibitor, such as, by way of example and by way of preference, ezetimibe, tiqueside or pamaqueside.

In a preferred embodiment of the invention, the fusion proteins according to the invention are administered in combination with an MTP inhibitor, such as, by way of example and by way of preference, implitapide, BMS-201038, R-103757 or JTT-130.

In a preferred embodiment of the invention, the fusion proteins according to the invention are administered in combination with a lipase inhibitor, such as, by way of example and by way of preference, orlistat.

In a preferred embodiment of the invention, the fusion proteins according to the invention are administered in combination with a thyroid hormone and/or thyroid mimetic, such as, by way of example and by way of preference. D-thyroxine or 3,5,3′-triiodothyronine (T3).

In a preferred embodiment of the invention, the fusion proteins according to the invention are administered in combination with an agonist of the niacin receptor, such GS. by way of example and by way of preference, niacin, acipimox, acifran or radecol.

In a preferred embodiment of the invention, the fusion proteins according to the invention are administered in combination with a CETP inhibitor, such as, by way of example and by way of preference, dalcetrapib. BAY 60-5521, anacetrapib or CETP vaccine (CETi-1).

In a preferred embodiment of the invention, the fusion proteins according to the invention are administered in combination with a PPAR-γ agonist, for example from the class of the thiazolidinediones, such as, by way of example and by way of preference, pioglitazone or rosiglitazone.

In a preferred embodiment of the invention, the fusion proteins according to the invention are administered in combination with a PPAR-δ agonist, such as, by way of example and by way of preference, GW-501516 or BAY 68-5042.

In a preferred embodiment of the invention, the fusion proteins according to the invention are administered in combination with a polymeric bile acid adsorber, such as, by way of example and by way of preference, cholestyramine, colestipol, colesolvam. CholestaGel or colestimide.

In a preferred embodiment of the invention, the fusion proteins according to the invention are administered in combination with a bile acid reabsorption inhibitor, such as, by way of example and by way of preference, ASBT (=IBAT) inhibitors, such as, for example, AZD-7806, S-8921, AK-105, BARI-1741, SC-435 or SC-635.

In a preferred embodiment of the invention, the fusion proteins according to the invention are administered in combination with an antioxidant/radical scavenger, such as, by way of example and by way of preference, probucol, AGI-1067. BO-653 or AEOL-10150.

In a preferred embodiment of the invention, the fusion proteins according to the invention are administered in combination with a cannabinoid receptor 1 antagonist, such as, by way of example and by way of preference, rimonabant or SR-147778.

Antidiabetics are to be understood as meaning, preferably, insulin and insulin derivatives, and also orally effective hypoglycemic active ingredients. Here, insulin and insulin derivatives include both insulins of animal, human or biotechnological origin and also mixtures thereof. The orally effective hypoglycemic active ingredients preferably include sulfonylureas, biguanides, meglitinide derivatives, glucosidase inhibitors and PPAR-gamma agonists.

In a preferred embodiment of the invention, the fusion proteins according to the invention are administered in combination with insulin.

In a preferred embodiment of the invention, the fusion proteins according to the invention are administered in combination with a sulfonylurea, such as, by way of example and by way of preference, tolbutamide, glibenclamide, glimepiride, glipizide or gliclazide.

In a preferred embodiment of the invention, the fusion proteins according to the invention are administered in combination with a biguanide, such as, by way of example and by way of preference, metformin.

In a preferred embodiment of the invention, the fusion proteins according to the invention are administered in combination with a meglitinide derivative, such as, by way of example and by way of preference, repaglinide or nateglinide.

In a preferred embodiment of the invention, the fusion proteins according to the invention are administered in combination with a glucosidase inhibitor, such as, by way of example and by way of preference, miglitol or acarbose.

In a preferred embodiment of the invention, the fusion proteins according to the invention are administered in combination with a DPP-IV inhibitor, such as, by way of example and by way of preference, sitagliptin and vildagliptin.

In a preferred embodiment of the invention, the fusion proteins according to the invention are administered in combination with a PPAR-gamma agonist, for example from the class of the thiazolinediones, such as, by way of example and by way of preference, pioglitazone or rosiglitazone.

The hypotensive agents are preferably understood as meaning compounds from the group of the calcium antagonists, angiotensin AII antagonists, ACE inhibitors, beta-receptor blockers, alpha-receptor blockers and diuretics.

In a preferred embodiment of the invention, the fusion proteins according to the invention are administered in combination with a calcium antagonist, such as, by way of example and by way of preference, nifedipine, amlodipine, verapamil or diltiazem.

In a preferred embodiment of the invention, the fusion proteins according to the invention are administered in combination with an angiotensin All antagonist, such as, by way of example and by way of preference, losartan, valsartan, candesartan, embusartan, olmesartan or telmisartan.

In a preferred embodiment of the invention, the fusion proteins according to the invention are administered in combination with an ACE inhibitor, such as, by way of example and by way of preference, enalapril, captopril, lisinopril, ramipril, delapril, fosinopril, quinopril, perindopril or trandopril.

In a preferred embodiment of the invention, the fusion proteins according to the invention are administered in combination with a beta-receptor blocker, such as, by way of example and by way of preference, propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the fusion proteins according to the invention are administered in combination with an alpha-receptor blocker, such as, by way of example and by way of preference, prazosin.

In a preferred embodiment of the invention, the fusion proteins according to the invention are administered in combination with a diuretic, such as, by way of example and by way of preference, furosemide, bumetanide, torsemide, bendroflumethiazide, chlorothiazide, hydrochlorothiazide, hydroflumethiazide, methyclothiazide, polythiazide, trichloromethiazide, chlorothalidone, indapamide, metolazone, quinethazone, acetazolamide, dichlorophenamide, methazolamide, glycerol, isosorbide, mannitol, amiloride or triamteren.

In a preferred embodiment of the invention, the fusion proteins according to the invention are administered in combination with an aldosterone or mineralocorticoid receptor antagonist, such as, by way of example and by way of preference, spironolactone or eplerenone.

In a preferred embodiment of the invention, the fusion proteins according to the invention are administered in combination with a vasopressin receptor antagonist, such as, by way of example and by way of preference, conivaptan, tolvaptan, lixivaptan or SR-121463.

In a preferred embodiment of the invention, the fusion proteins according to the invention are administered in combination with an organic nitrate or NO donor, such GS. by way of example and by way of preference, sodium nitroprusside, nitroglycerol, isosorbide mononitrate, isosorbide dinitrate, molsidomin or SIN-1, or in combination with inhalative NO.

In a preferred embodiment of the invention, the fusion proteins according to the invention are administered in combination with a positive-inotropic compound, such as, by way of example and by way of preference, cardiac glycosides (digoxin), beta-adrenergic and dopaminergic agonists, such as isoproterenol, adrenaline, noradrenaline, dopamine or dobutamine.

In a preferred embodiment of the invention, the fusion proteins according to the invention are administered in combination with antisympathotonics, such as reserpine, clonidine or alpha-methyldopa, or in combination with potassium channel agonists, such as minoxidil, diazoxide, dihydralazine or hydralazine, or with substances which release nitrogen oxide, such as glycerol nitrate or sodium nitroprusside.

Antithrombotics are to be understood as meaning, preferably, compounds from the group of the platelet aggregation inhibitors or the anticoagulants.

In a preferred embodiment of the invention, the fusion proteins according to the invention are administered in combination with a platelet aggregation inhibitor, such as, by way of example and by way of preference, aspirin, clopidogrel, ticlopidine or dipyridamol.

In a preferred embodiment of the invention, the fusion proteins according to the invention are administered in combination with a thrombin inhibitor, such as, by way of example and by way of preference, ximelagatran, melagatran, dabigatran, bivalirudin or clexane.

In a preferred embodiment of the invention, the fusion proteins according to the invention are administered in combination with a GPIIb/IIIa antagonist, such as, by way of example and by way of preference, tirofiban or abciximab.

In a preferred embodiment of the invention, the fusion proteins according to the invention are administered in combination with a factor Xa inhibitor, such as, by way of example and by way of preference, rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428, provided that the PCS is not a factor Xa cleavage site.

In a preferred embodiment of the invention, the fusion proteins according to the invention are administered in combination with heparin or a low molecular weight (LMW) heparin derivative.

In a preferred embodiment of the invention, the fusion proteins according to the invention are administered in combination with a vitamin K antagonist, such as, by way of example and by way of preference, coumarin.

In the context of the present invention, particular preference is given to combinations comprising at least one of the fusion proteins according to the invention and also one or more further active ingredients selected from the group consisting of HMG-CoA reductase inhibitors (statins), diuretics, beta-receptor blockers, organic nitrates and NO donors, ACE inhibitors, angiotensin All antagonists, aldosterone and mineralocorticoid receptor antagonists, vasopressin receptor antagonists, platelet aggregation inhibitors and anticoagulants, and also their use for the treatment and/or prevention of the disorders mentioned above.

The present invention furthermore provides medicaments comprising at least one fusion protein according to the invention, usually together with one or more inert nontoxic pharmaceutically suitable auxiliaries, and also their use for the purposes mentioned above.

### Therapeutically Effective Dose

Pharmaceutical compositions suitable for use in the present invention include compositions wherein the active ingredients are contained in an effective amount to achieve the intended purpose, e.g. heart failure. The determination of an effective dose is well within the capability of those skilled in the art. For any compound, the therapeutically effective dose can be estimated initially either in in vitro assays, e.g. LGR7 receptor activation, ex vivo in isolated perfused rat hearts, or in animal models, usually mice, rabbits, dogs, or pigs. The animal model is also used to achieve a desirable concentration range and route of administration. Such information can then be used to determine useful doses and routes for administration in humans.

A therapeutically effective dose refers to that amount of fusion protein that ameliorates the symptoms or condition. Therapeutic efficacy and toxicity of such compounds can be determined by standard pharmaceutical procedures in vitro or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, ED50/LD50. Pharmaceutical compositions that exhibit large therapeutic indices are preferred. The data obtained from in vitro assays and animal studies are used in formulating a range of dosage for human use. The dosage of such compounds lies preferably within a range of circulating concentrations what include the ED50 with little or no toxicity. The dosage varies within this range depending upon the dosage form employed, sensitivity of the patient, and the route of administration.

Normal dosage amounts may vary from 0.1 to 100,000 milligrams total dose, depending upon the route of administration. Guidance as to particular dosages and methods of delivery is provided in the literature. See U.S. Pat. No. 4,657,760; 5,206,344; or 5,225,212. Those skilled in the art will employ different formulations for polynucleotides than for proteins or their inhibitors. Similarly, delivery of polynucleotides or polypeptides will be specific to particular cells, conditions, locations, etc.

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention. All examples were carried out using standard techniques, which are well known and routine to those of skill in the art, except where otherwise described in detail. Routine molecular biology techniques of the following examples can be carried out as described in standard laboratory manuals, such as Sambrook et al., Molecular Cloning: A Laboratory Manual, 2nd Ed.; Cold Spring Harbor Laboratory Press, Cold Spring Harbor, N.Y., 1989.

Further preferred embodiments are:
1. A fusion protein comprising Relaxin-PCS-HEM or HEM-PCS-Relaxin, wherein
   Relaxin comprises a Relaxin A chain polypeptide or a functional variant thereof,
      and a Relaxin B chain polypeptide or a functional variant thereof.
   PCS comprises an endo-protease cleavage site, and
   HEM is a proteinaceous half-life extending moiety.
2. A fusion polypeptide comprising proRelaxin-PCS-HEM or HEM-PCS-proRelaxin,
   wherein
   proRelaxin comprises a Relaxin A chain polypeptide or a functional variant thereof,
   a relaxin C-chain polypeptide and a Relaxin B chain polypeptide or a functional variant thereof.
   PCS comprises an endo-protease cleavage site, and
   HEM is a proteinaceous half-life extending moiety.
3. A fusion protein Cr polypeptide according to counts 1 or 2, wherein the PCS is a cleavage site of an extracellular endo-protease.
4. A fusion protein or polypeptide according to count 3, wherein the endo-protease is an endogenous endo-protease.
5. A fusion protein or polypeptide according to counts 3 or 4, wherein the endo-protease is a cardiac, liver, kidney or lung expressed endo-protease.
6. A fusion protein or polypeptide according to count 3, wherein the endo-protease is a membrane bound or membrane spanning protease having its catalytic activity on the extracellular side of the membrane.
7. A fusion protein Cr polypeptide according to anyone of counts 1 to 6, wherein the endo-protease is selected from the group of endoproteases represented by table 1.
8. A fusion protein or polypeptide according to anyone of counts 1 to 6, wherein the PCS is selected from the group of PCS represented by table 1.
9. A fusion protein or polypeptide according to anyone of counts 3-8, wherein the endo-protease is selected from the group consisting of factor Xa, Trypsin, MMP2, MMP9, MMP12, Renin, Elastase and Chymase.
10. A fusion protein or polypeptide according to anyone of counts 3-9, wherein the endo-protease is human.
11. A fusion protein or polypeptide according to anyone of counts 1-10, wherein the PCS has a sequence comprised in a group of sequences consisting of IleGluGlyArgMetAsp (FXa cleavage site), RAKRFASL (MMP9 cleavage site), INARVSTI (Trypsin cleavage site), RVGFYESD (Chymase cleavage site) and GLRVGFYE (Elastase cleavage site).
12. A fusion protein or polypeptide according to anyone of counts 1-11, wherein the PCS has a stretcher polypeptide at the N-terminus and/or at the C-terminus.
13. A fusion polypeptide according to anyone of the foregoing counts, wherein the proteinaceous half-life extending moieties are comprised in a group of proteinaceous half-life extending moieties consisting of immunoglobulin Fc domain, serum albumin, transferrin and serum albumin binding protein.
14. A fusion polypeptide according to anyone of the foregoing counts, wherein the proteinaceous half-life extending moiety is an IgG1 Fc domain.
15. A fusion polypeptide according to anyone of the foregoing counts, wherein the proteinaceous half-life extending moiety is human.
16. A fusion polypeptide according to anyone of the foregoing counts, wherein the Relaxin A chain is human Relaxin 2 A chain and the Relaxin B chain is human Relaxin 2 B chain.
17. A fusion polypeptide according to anyone of the foregoing counts, wherein the fusion polypeptide is proRelaxin-PCS-HEM.
18. A fusion protein according to anyone of the foregoing counts, wherein the fusion polypeptide is Relaxin-PCS-HEM.
19. A polynucleotide encoding a proRelaxin-PCS-HEM or HEM-PCS-proRelaxin fusion polypeptide according anyone of counts 2-18.
20. A vector comprising a polynucleotide according to count 19.
21. A host cell comprising a vector according to count 20 or a polynucleotide according to count 17.
22. A method of producing a Relaxin-PCS-HEM or HEM-PCS-Relaxin protein according to anyone of counts 1-18 comprising the steps of cultivating a host cell of count 21 further comprising a prohormone convertase activity and isolating the protein.
23. A pharmaceutical composition comprising a Relaxin-PCS-HEM or HEM-PCS-Relaxin protein according to anyone of counts 1-18.
24. A pharmaceutical composition according to count 23 or a Relaxin-PCS-HEM or HEM-PCS-Relaxin protein according to anyone of counts 1-18 as medicament.
25. A pharmaceutical composition according to count 23 or 24 or a Relaxin-PCS-HEM or HEM-PCS-Relaxin protein according to anyone of counts 1-18 as medicament for the treatment of cardiovascular disease, lung disease, fibrotic disorder or kidney disease.
26. A method of treating a cardiovascular disease, lung disease, fibrotic disorder or kidney disease comprising the administration of a therapeutically effective dose of a pharmaceutical composition according to count 23 and 24 or a Relaxin-PCS-HEM or HEM-PCS-Relaxin protein according to anyone of counts 1-18.
27. A treatment according to counts 25 or 26, wherein the cardiovascular disease is comprised in the group of cardiovascular diseases consisting of coronary heart disease, acute coronary syndrome, heart failure, or myocardial infarction.

### EXAMPLES

### Experimental Protocols

### Construction of Relaxin-Fc Fusion Proteins:

The cDNA sequences of the Relaxin variants were generated by chemical gene synthesis. The synthesized genes were subcloned into the mammalian expression vector pCEP4 (Invitrogen, catalogue number VO44-50). As signal leader sequence for correct secretion of the resulting protein, either the leader sequence of the LDL receptor-related protein (LRP, amino acid composition MLTPPLLLLLPLLSALVAA) or of CD33 (amino acid composition MPLLLLLPLLWAGALA) were used. For subcloning of the synthesized constructs the restriction enzymes HindIII and BamH1 were used according to manufactures' instruction.

To improve the plasma half life the Fc part of the human IgG1 was combined with human Relaxin 2 by chemically based gene synthesis. The carboxy-terminal part of human Relaxin 2 (according to its genomic organization arranged as follows: B chain-C chain-A chain) was fused to N terminal end of the human IgG1 Fc moiety, whereby these two parts of the fusion protein were connected by a 6 amino acids long linker sequence consisting of a polypeptide with the sequence IleGluGlyArgMetAsp encoding the coagulation factor Xa cleavage site.

The proRelaxin-Fc fusion has the following sequence (protein: SEQ ID NO: 1: nucleotide sequence: SEQ ID NO: 17):

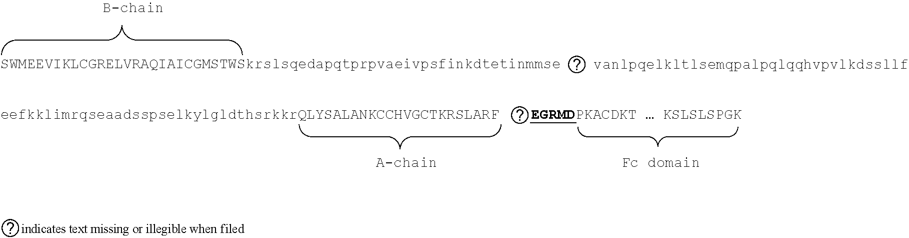

The C-chain sequence, which is excised by the pro-hormone convertase, is denoted in small letters. The FXa cleavge site is marked in bold, underlined letters.

Another option to improve the biological half life of polypeptides are fusions with polypeptides like Transferrin (accession number P02787) or Albumin (accession number P02768). (SR Schmid (2009)).

Another option is the usage of other protease cleavage sites than the one for FXa, e.g. cleavage sites listed up in table 1.

The construct Relaxin-Fusion 1 exhibiting a MMP9 cleavage site has SEQ ID NO: 13 (polypeptide) and the nucleotide sequence SEQ ID NO. 29.

The construct Relaxin-Fusion 2 exhibiting a Chymase cleavage site has SEQ ID NO: 14 (polypeptide) and the nucleotide sequence SEQ ID NO. 30.

The construct Relaxin-Fusion 3 exhibiting a Trypsin cleavage site has SEQ ID NO: (polypeptide) and the nucleotide sequence SEQ ID NO. 31.

The construct Relaxin-Fusion 4 exhibiting a Elastase cleavage site has SEQ ID NO: 16 (polypeptide) and the nucleotide sequence SEQ ID NO. 32.

### Expression of Relaxin Fc Fusion Proteins:

For small scale expression (up to 2 milliliter culture volume) HEK293 (ATCC, catalogue number CRL-1573) cells were transiently transfected with the expression plasmid encoding the Relaxin-Fc fusion construct using Lipofectamine-2000 Transfection Reagent (Invitrogen, catalogue number 11668-019) according to manufactures' Instructions. For correct processing of the Relaxin, cells were co-transfected with an expression vector encoding the human Prohormone Convertase 1 (accession number NP_{—}000430.3). Cells were cultivated in D-Mem F12 (Gibco, #31330), 1% Penicillin-Streptomycin (Gibco, #15140) and 10% fetal calf serum (FCS, Gibco, #11058) in a hunified incubator at 5% carbon dioxide at 37° C. Three to five days following transfection, conditioned medium of the transfected cells were tested for activity using the stably transfected CHO-CRE-GR7 cell line.

For large scale expression (10 milliliter culture volume and more) the constructs were transiently expressed in mammalian cell cells as described in Tom et al., 2007. Briefly, the expression plasmid transfected into HEK293-6E cells and incubated in Fernbach-Flasks or Wave-Bags. Expression was at 37° C. for 5 to 6 days in F17 Medium (Invitrogen). 5 g/l Tryptone TN1 (Organotechnie), 1% Ultra-Low IgG FCS (Invitrogen) and 0.5 mM Valproic acid (Sigma) were supplemented after transfection.

### Purification of Relaxin Fc Fusion Protein:

Relaxin Fc-Fusion constructs are purified from mammalian cell culture supernatants. First supernatants are clarified from cell debris by centrifugation. Proteins are purified by Protein A (MabSelect Sure, GE Healthcare) affinity chromatography followed by size exclusion chromatography (SEC). Therefore the supernatant is applied to a Protein A column previously equilibrated in PBS pH 7.4 (Sigma/Aldrich), contaminants are removed with 10 column volumes of PBS pH 7.4+500 mM NaCl. Relaxin Fc Fusion constructs are eluted with 50 mM Na-acetate pH 3.5+500 mM NaCl and further purified by SEC on a Superdex 200 column in PBS pH 7.4.

### Quantification of Expressed Relaxin-Fc Fusion Proteins:

For quantification of secreted and purified recombinant Relaxin variants, the commercially available quantification ELISA (R&D Systems, Human Relaxin-2 Quantikine ELISA Kit, catalogue number DRL200) was used according to the manufactures' instructions.

In addition for some constructs proteins were quantified by using FC-ELISA. For the Fc ELISA, 96 well microtitter plates (Nunc, Maxi Sorp black, catalogue number 460918) were coated with an anti-Fc antibody (SigmaAldrich, catalogue number A2136) over night at 4° C. and a concentration of 5 μg per milliliter. Plates were washed once by using 50 microliter per well of a buffer consisting of PBS and 0.05% Tween 20 (SigmaAldrich, catalogue number 63158) buffer. Thirty microliter of a blocking buffer (Candor Bioscience, catalogue number 113500) was added and the plate incubated for 1 hour at 37° C. Plates were washed 3 times using 50 microliter per well of the PBS/0.05% Tween 20 buffer. Samples were added and the plates incubated were for 1 hour at 37° C. If necessary, samples have to be diluted by using the above mentioned blocking buffer. After incubation, plates were washed 3 times using 50 microliter per well of the PBS/0.05% Tween 20 buffer.

For detection 30 microliter of a Anti-h-Fc-POD (SigmaAldrich, catalogue number A0170) diluted 1:10000 in 10% blocking buffer was added and incubated for 1 hour at 37° C. After incubation, plates were washed 3 times using 50 microliter per well of the PBS/0.05% Tween 20 buffer. Thirty microliter of BM Blue Substrate POD (Roche Diagnostics, catalogue number 11484281001) was added and after five minutes of incubation, the reaction was stopped by the addition of a 1 molar acid sulfur solution. Absorption was measured using the Tecan Infinite 500 reader, absorbance mode, extinction 450 nm, emission 690 nm.

### Activity Testing:

CHO K1 cells (ATCC, catalogue number CCL-61) were stably transfected with the cyclic AMP responsive element (CRE) Luciferase reporter gene construct (Biomyx Technology, pHTS-CRE, catalogue number P2100) resulting in a CHO-CRE-Luciferase cell line.

This cell line was subsequently stably transfected with the human LGR7/RXFP1 receptor (accession numbers NM_{—}021634.2), cloned as 2271 base pair long DNA fragment into the mammalian expression vector pcDNA3.1(−) (Invitrogen, catalogue number V79520), resulting in a CHO-CRE-LGR7 cell line. This cell line was cultivated in D-Mem F12 (Gibco, #31330) 2 mM Glutamax (Gibco, #35050), 100 nM Pyruvat (Gibco, #11360-070), 20 mM Hepes (Gibco, #15630), 1% Penicillin-Streptomycin (Gibco, #15140) and 10% fetal calf serum (FCS, Gibco, #11058).

For stimulation, medium was exchanged by OptiMem (Gibco, #11058)+1% FCS containing different concentrations of the recombinantly expressed Relaxin-Fc fusion proteins (usually starting at a concentration of 100 nM, followed by 1:2 dilutions). As positive control, commercially available recombinant expressed human Relaxin 2 was used (R&D Systems, catalogue number 6586-RN-025). Subsequently, cells were incubated for 6 hours in a hunified incubator at 5% carbon dioxide at 37° C. After 6 hours cells were tested for Luciferase activity using a Luciferase Assay System (Promega, #E1500) and using the Tecan Infinite 500 reader, luminescence mode, 1000 milliseconds integration time, measurement time 30 seconds.

Relative luminescence units were used to determine EC50 values of the different molecules by using the computer program Graph Pad Prism Version 5.

For alternative activity testing of Relaxin as well as of fusion polypeptides of the invention, cell lines (e.g. THP1, ATCC catalogue number TIB-202) or primary cells (e.g. Celprogen Inc., Human Cardiomyocyte Cell Culture, catalogue number 36044-15) with endogenous expression of the LGR7 receptor are used. These cells are cultivated according to the manufactures instruction.

Methods for the detection of Relaxin or Relaxin-Fc fusion proteins induced generation of cAMP are known in the art. For example, such measurement is performed using a cAMP ELISA (e.g. IBL International GmbH, cAMP ELISA, catalogue number CM 581001) according to the manufactures instruction.

Methods for the detection of Relaxin or Relaxin-Fc fusion proteins induced activation of PI3 kinase are known in the art. For example, such measurement is performed using a PI3-Kinase HTRF Assay according to the manufactures instruction (e.g. Millipore, PI3-Kinase HTRF Assay, catalogue number 33-016).

### Protease Treatment of Relaxin-Fc Fusion Proteins and Activity Testing.

Supernatants of HEK293 cells expressing the Relaxin-Fusion proteins are incubated with the corresponding proteases indicated as follows:
2 ml supernatant of HEK293 cells expressing Relaxin-Fc were incubated with 1 μg of Factor Xa Protease (New England Biolabs, catalogue number P8010) for 6 hours at 23° C.
2 ml supernatant of HEK293 cells expressing Relaxin-Fusion 2 were incubated at a concentration of 0.83 μg/ml of Chymase (Sigma Aldrich, catalogue number C8118) for 6 hours at 37° C.
2 ml supernatant of HEK293 cells expressing Relaxin-Fusion 3 were incubated at a concentration of 10 μg/ml of Trypsin (Sigma Aldrich, catalogue number T0303) for 6 hours at 37° C.
2 ml supernatant of HEK293 cells expressing Relaxin-Fusion 4 were incubated at a concentration of 5 μg/ml of Elastase (Sigma Aldrich, catalogue number E7885) for 6 hours at 37° C.
Before usage, MMP9 (R&D Systems, catalogue number 911-MP) has to be activated by incubating the protease with APMA (p-aminophenylmercuric acetate; Sigma Aldrich, catalogue number A-9563). For this, MMP9 has to be diluted in Assay Buffer (50 mM Tris, 10 mM CaCl2, 150 mM NaCl2, 0.05% Brij35, pH 7.5.) to a concentration of 100 μg/ml (e.g. 1 μg in a final volume of 100 μl). APMA is added to a final concentration of 1 mM (e.g. 20 μl of a 5 mM stock solution in a final volume of 100 μl). This mixture is incubated for 24 hours at 37° C. Afterwards, the activated MMP9 is diluted in 2 ml supernatant of HEK293 cells expressing the Relaxin-Fusion 1 to a final concentration of 0.4 ng/ml. were incubated with the activated MMP9 for 6 hours at 37° C.

Supernatants of Relaxin-Fc fusion protein expressing HEK293 cells were tested for activity by using the CHO-CRE-LGR7 cell line as described above. As positive control, human Relaxin 2 was used.

For the Relaxin-Fc fusion protein, no activity was detected. In contrast, after FXa incubation of the Relaxin-Fc fusion protein containing supernatant, significant activation of the CHO-CRE-LGR7 cell line was observed. Although this activity was lower than the activity obtained for the human Relaxin 2 positive control it shows that with the employment of a PCS a releasable active Relaxin molecule was generated. Use of non-purified Relaxin-Fusion proteins is an likely explanation of the slightly lower activity as possible impurities in the sample leads to false determination of the concentration or could have a negative impact on the accuracy of the cell based Luciferase assay.

Using supernatants of HEK293 cells transfected with the empty expression vector leads to a reduction in the activity assay by a factor of approximately 3. Another explanation could be incomplete cleavage of the Relaxin-Fusion proteins leading to a mixture of cleaved off and functional active Relaxin and inactive Relaxin-Fusion proteins.

### Example 1

### Relaxin-Fc

To improve the biological half life the Fc part of the human IgG1 was combined with human Relaxin 2 by chemically based gene synthesis. The carboxy-terminal part of human Relaxin 2 (according to its genomic organization arranged as follows: B chain-C chain-A chain) was fused to N terminal end of the human IgG1 Fc moiety, whereby these two parts of the fusion protein were connected by a 6 amino acids long linker sequence consisting of a polypeptide with the sequence IleGluGlyArgMetAsp encoding the coagulation factor Xa cleavage site. Relaxin only shows detectable activity by using the CHO-CRE-LGR7 cell line after incubating the construct with the protease FXa as described above.

### Example 2

### Relaxin-Fusion 1

To improve the biological half life the Fc part of the human IgG1 was combined with human Relaxin 2 by chemically based gene synthesis. The carboxy-terminal part of human Relaxin 2 (according to its genomic organization arranged as follows: B chain-C chain-A chain) was fused to N terminal end of the human IgG1 Fc moiety, whereby these two parts of the fusion protein were connected by a 6 amino acids long linker sequence consisting of a polypeptide with the sequence ArgAlaLysArgPheAlaSerLeu encoding the protease MMP9 cleavage site. Relaxin only shows detectable activity by using the CHO-CRE-LGR7 cell line after incubating the construct with the protease MMP9 as described above.

### Example 3

### Relaxin-Fusion 2

To improve the biological half life the Fc part of the human IgG1 was combined with human Relaxin 2 by chemically based gene synthesis. The carboxy-terminal part of human Relaxin 2 (according to its genomic organization arranged as follows: B chain-C chain-A chain) was fused to N terminal end of the human IgG1 Fc moiety, whereby these two parts of the fusion protein were connected by a 6 amino acids long linker sequence consisting of a polypeptide with the sequence ArgValGlyPheTyrGluSerAsp encoding the protease Chymase cleavage site. Relaxin only shows detectable activity by using the CHO-CRE-LGR7 cell line after incubating the construct with the protease Chymase as described above. Low signal values obtained in the Chymase experiment could be due to cleavage of the LGR7 receptor expressed by the screening cell line by the added Chymase Protease. The skilled person in the art knows how to remove or reduce Chymase activity in the assay system (e.g. use of specific protease inhibitors). Nevertheless, these data demonstrate that functional Relaxin can be released from the fusion protein.

### Example 4

### Relaxin-Fusion 3

To improve the biological half life the Fc part of the human IgG1 was combined with human Relaxin 2 by chemically based gene synthesis. The carboxy-terminal part of human Relaxin 2 (according to its genomic organization arranged as follows: B chain-C chain-A chain) was fused to N terminal end of the human IgG1 Fc moiety, whereby these two parts of the fusion protein were connected by a 6 amino acids long linker sequence consisting of a polypeptide with the sequence IleAsnAlaArgValSerThrlle encoding the protease Trypsin cleavage site. Relaxin only shows significant activity after incubating the supernatant with Trypsin as described above. The non-incubated supernatant shows minor activity, possibly due to protease contaminants in the cell culture supernatants, which recognizes similar cleavage sites than Trypsin.

### Example 5

### Relaxin-Fusion 4

To improve the biological half life the Fc part of the human IgG1 was combined with human Relaxin 2 by chemically based gene synthesis. The carboxy-terminal part of human Relaxin 2 (according to its genomic organization arranged as follows: B chain-C chain-A chain) was fused to N terminal end of the human IgG1 Fc moiety, whereby these two parts of the fusion protein were connected by a 6 amino acids long linker sequence consisting of a polypeptide with the sequence GlyLeuArgValGlyPheTyrGlu encoding the protease Elastase cleavage site. Relaxin only shows detectable activity by using the CHO-CRE-LGR7 cell line after incubating the construct with the protease Elastase as described above. The non-incubated supernatant shows minor activity, possibly due to protease contaminants in the cell culture supernatants, which recognizes similar cleavage sites than Elastase.

TABLE 2
A list of constructs and corresponding SEQ ID NOs.

| Name | Description | SEQ ID NO: |
|---|---|---|
| Relaxin-Fc | Relaxin-Fxa cleavage site-hum | SEQ ID NO: 1 |
| | IgG1 Fc | |
| Transferrin | Transferrin | SEQ ID NO: 2 |
| Albumin | Albumin | SEQ ID NO: 3 |
| Fc IgG1 human | Fc IgG1 human | SEQ ID NO: 4 |
| human Relaxin 2 | human Relaxin 2 | SEQ ID NO: 5 |
| RLN2 A chain | RLN2 A chain | SEQ ID NO: 6 |
| RLN2 minimal A | RLN2 minimal A chain | SEQ ID NO: 7 |
| chain | | |
| RLN2 B chain | RLN2 B chain | SEQ ID NO: 8 |
| human Relaxin 3 | human Relaxin 3 | SEQ ID NO: 9 |
| RLN3 A chain | RLN3 A chain | SEQ ID NO: 10 |
| RLN3 B chain | RLN3 B chain | SEQ ID NO: 11 |
| RLN3 minimal A | RLN3 minimal A chain | SEQ ID NO: 12 |
| chain | | |
| Relaxin-Fusion 1 | Relaxin-MMP9 cleavage site- | SEQ ID NO: 13 |
| | humFc IgG1 | |
| Relaxin-Fusion 2 | Relaxin-Chymase cleavage site- | SEQ ID NO: 14 |
| | humFc IgG1 | |
| Relaxin-Fusion 3 | Relaxin-Trypsin cleavage site- | SEQ ID NO: 15 |
| | humFc IgG1 | |
| Relaxin-Fusion 4 | Relaxin-Elastase cleavage site- | SEQ ID NO: 16 |
| | humFc IgG1 | |
| Relaxin-Fc | Relaxin-Fxa cleavage site-hum | SEQ ID NO: 17 |
| | IgG1 Fc | |
| Transferrin | Transferrin | SEQ ID NO: 18 |
| Albumin | Albumin | SEQ ID NO: 19 |
| Fc IgG1 human | Fc IgG1 human | SEQ ID NO: 20 |
| human Relaxin 2 | human Relaxin 2 | SEQ ID NO: 21 |
| RLN2 A chain | RLN2 A chain | SEQ ID NO: 22 |
| RLN2 minimal A | RLN2 minimal A chain | SEQ ID NO: 23 |
| chain | | |
| RLN2 B chain | RLN2 B chain | SEQ ID NO: 24 |
| human Relaxin 3 | human Relaxin 3 | SEQ ID NO: 25 |
| RLN3 A chain | RLN3 A chain | SEQ ID NO: 26 |
| RLN3 B chain | RLN3 B chain | SEQ ID NO: 27 |
| RLN3 minimal A | RLN3 minimal A chain | SEQ ID NO: 28 |
| chain | | |
| Relaxin-Fusion 1 | Relaxin-MMP9 cleavage site- | SEQ ID NO: 29 |
| | humFc IgG1 | |
| Relaxin-Fusion 2 | Relaxin-Chymase cleavage site- | SEQ ID NO: 30 |
| | humFc IgG1 | |
| Relaxin-Fusion 3 | Relaxin-Trypsin cleavage site- | SEQ ID NO: 31 |
| | humFc IgG1 | |
| Relaxin-Fusion 4 | Relaxin-Elastase cleavage site- | SEQ ID NO: 32 |
| | humFc IgG1 | |

### CITATIONS

Arlaud, G. J., Rossi, V., Gaboriaud, C. and Thielens, N. M. Complement component C1s. In Handbook of Proteolytic Enzymes, 2 edn (Barrett, A. J., Rawlings, N. D. and Woessner, J. F. eds), p. 1620-1623, Elsevier, London (2004)
auf dem Keller, U., Prudova, A., Gioia, M., Butler, G. S, and Overall, C. M. (2010) A statistics-based platform for quantitative N-terminome analysis and identification of protease cleavage products. Mol Cell Proteomics 9:912-927
Ausubel, F. M. et al. (eds.) Current Protocols in Molecular Biology, Greene Publishing Associates, (1989)
Bani, D., Masini, E., Bello, M. G., Bigazzi, M. and Sacchi, T. B. (1998) Relaxin protects against myocardial injury caused by ischemia and reperfusion in rat heart. Am. J. Pathol. 152:1367-1376
Bani-Sacchi, T., Bigazzi, M., Bani, D., Mannaioni, P. F., and Masini, E. (1995) Relaxin-induced increased coronary flow through stimulation of nitric oxide production. Br J Pharmacol 116:1589-1594
Barbs K K, Gatos D, Vasileiou Z, Barbs K. (2010) An optimized chemical synthesis of human relaxin-2. J Pept Sci. 16:200-211
Bartsch, O., Bartlick, B., and Nell, R. (2001). Relaxin signaling links tyrosine phosphorylation to phosphodiesterase and adenylyl cyclase activity. Mol Hum Reprod 7:799-809
Bartsch, O., Bartlick, B., and Nell, R. (2004). Phosphodiesterase 4 inhibition synergizes with relaxin signaling to promote decidualization of human endometrial stromal cells. J Clin Endocrinol Metab 89:324-334
Behrens, Spiekerman and Brown (1975) Fed. Proc. Fed. Am. Soc. Exp. Biol. 34, 591 Bennett R G. (2009) Relaxin and its role in the development and treatment of fibrosis. Transl Res. 154:1-6
Benton T. Chen T. McEntee M. Fox B. King O. Crombie R. Thomas T C, Bebbington C. (2002) The use of UCOE vectors in combination with a preadapted serum free, suspension cell line allows for rapid production of large quantities of protein. Cytotechnology 38:43-46
Bjork, I., Danielsson, A., Fenton, J. W. and Jornvall (1981) The site in human antithrombin for functional proteolytic cleavage by human thrombin. FEBS Lett 126:257-260.
Büllesbach E E, Schwabe C. (2000) The relaxin receptor-binding site geometry suggests a novel gripping mode of interaction. J Biol. Chem. 275:35276-35280 Castrop H, Höcherl K, Kurtz A, Schweda F, Todorov V, Wagner C. (2010) Physiology of kidney renin. Physiol Rev. 2010 90:607-673
Caughey, G. H., Raymond, W. W. and Wolters, P. J. (2000) Angiotensin II generation by mast cell alpha- and beta-chymases. Biochim Biophys Acta 1480:245-257
Cirman, T., Oresic, K., Mazovec, G. D., Turk, V., Reed, J. C., Myers, R. M., Salvesen, G. S, and Turk, B. (2004) Selective disruption of lysosomes in HeLa cells triggers apoptosis mediated by cleavage of Bid by multiple papain-like lysosomal cathepsins. J Biol Chem 279:3578-3587
Colligan, Current Protocols in Immunology, or Current Protocols in Protein Science, John Wiley & Sons, N Y, N. Y., (1997-2001), e.g., Chapters 1, 4, 6, 8, 9, 10
Cosen-Binker L I, Binker M G, Cosen R. Negri G. Tiscornia O. (2006) Relaxin prevents the development of severe acute pancreatitis. World J. Gastroenterol. 12:1558-1568; Coward W R, Saini G, Jenkins G. (2010) The pathogenesis of idiopathic pulmonary fibrosis. Ther Adv Respir Dis. 4:367-388
Dennis M S, Zhang M. Meng Y G. Kadkhodayan M. Kirchhofer D, Combs D, Damico L A. (2002) Albumin binding as a general strategy for improving the pharmacokinetics of proteins. J Biol. Chem. 277:35035-35043
Dschietzig T. Bartsch C. Baumann G. Stangl K. (2006) Relaxin—a pleiotropic hormone and its emerging role for experimental and clinical therapeutics. Pharmacol. Ther. 112:38-56
Dschietzig T. Teichmann G. Unemori E. Wood G. Boehmer J, Richter C, Baumann G. Stangl K (2009) Intravenous Recombinant Human Relaxin in Compensated Heart Failure: A Safety, Tolerability, and Pharmacodynamic Trial. J Cardiac Fail 5:182-190
Duncan, R. C., Wijeyewickrema, L. C. and Pike, R. N. (2008) The initiating proteases of the complement system: controlling the cleavage. Biochimie 90:387-395
Dunn, B. M. (2004) Rhizopuspepin. In Handbook of Proteolytic Enzymes, 2 edn (Barrett, A. J., Rawlings, N. D. & Woessner, J. F. eds), p. 108-111, Elsevier, London
Durocher Y, Ferret S. Kamen A. (2002) High-level and high-throughput recombinant protein production by transient transfection of suspension-growing human 293-EBNA1 cells. Nucleic Acids Res. 15; 30(2):E9
Edelstein, C., Italia, J. A., Klezovitch, O. and Scanu, A. M. (1996) Functional and metabolic differences between elastase-generated fragments of human lipoprotein[a] and apolipoprotein. J Lipid Res 37:1786-1801
Fosang, A. J., Last, K., Fujii, Y., Seiki, M. and Okada, Y. (1998) Membrane-type 1 MMP (MMP-14) cleaves at three sites in the aggrecan interglobular domain. FEBS Lett 430:186-190
Fosang, A. J., Neame, P. J., Last, K., Hardingham, T. E., Murphy, G. and Hamilton, J. A. (1992) The interglobular domain of cartilage aggrecan is cleaved by PUMP, gelatinases, and cathepsin B. J Biol Chem 267:19470-19474
Freeman W H and Co. (1992) Antibody Engineering, A Practical Guide, Borrebaeck, C. A. K., ed.
Garbacki N. Di Valentin E. Piette J. Cataldo O. Crahay C. Colige A. (2009) Matrix metalloproteinase 12 silencing: a therapeutic approach to treat pathological lung tissue remodeling? Pulm Pharmacol Ther. 22:267-278
Goeddel; (1990) Gene Expression Technology. Methods in Enzymology 185, Academic Press, San Diego, Calif.
Haas, C., Aldudo, J., Cazorla, P., Bullido, M. J., de Miguel, C., Vazquez, J. and Valdivieso, F. (1997) Proteolysis of Alzheimer's disease beta-amyloid precursor protein by factor Xa. Biochim Biophys Acta 1343:85-94
Halls M. L., Bathgate R. A., Summers, R. J. (2005) Signal Switching after Stimulation of LGR7 Receptors by Human Relaxin 2. Ann. N. Y. Acad. Sci. 1041:288-291
Harris C L, Hughes C E, Williams A S, Goodfellow I, Evans D J, Caterson B. and Morgan B P (2003) Generation of Anti-complement “Prodrugs”: J. Biol. Chem. 278: 36068-36076
Harris J M, Martin N E, Modi M. (2001) Pegylation: a novel process for modifying pharmacokinetics. Clin Pharmacokinet. 40:539-551
Hossain M A, Rosengren K J, Haugaard-Jonsson L M, Zhang S, Layfield G. Ferraro T, Daly N L, Tregear G W, Wade J D, Bathgate R A. (2008) The A-chain of human relaxin family peptides has distinct roles in the binding and activation of the different relaxin family peptide receptors. J Biol. Chem. 283:17287-17297
Hsu, S. Y. (2003). New insights into the evolution of the relaxin-LGR signaling system. Trends Endocrinol Metab 14:303-309
Hudson P. Haley J, John M. Cronk M. Crawford R. Haralambidis J, Tregear G, Shine J. Niall H. (1983) Structure of a genomic clone encoding biologically active human relaxin. Nature 301: 628-631
Ireland, L. S., Harrison, D. J., Neal, G. E. and Hayes, J. D. (1998) Molecular cloning, expression and catalytic activity of a human AKR7 member of the aldo-keto reductase superfamily: evidence that the major 2-carboxybenzaldehyde reductase from human liver is a homologue of rat aflatoxin B1-aldehyde reductase. Biochem J 332:21-34
Johnson, G. D. and Bond, J. S. (1997) Activation mechanism of meprins, members of the astacin metalloendopeptidase family. J Biol Chem 272:28126-28132
Kenneth, A et al: Chemical Stability of Pharmaceuticals: A Handbook for Pharmacists
Klein J. Kavvadas P. Prakoura N. Karagianni F. Schanstra J P, Bascands J L, Charonis A. (2011) Renal fibrosis: insight from proteomics in animal models and human disease. Proteomics. 11:805-815
Kong R C, Shilling P J, Lobb D K, Gooley P R, Bathgate R A. (2010) Membrane receptors: structure and function of the relaxin family peptide receptors. Mol Cell Endocrinol. 320):1-15
Lam, J. K., Chion, C. K., Zanardelli, S., Lane, D. A. and Crawley, J. T. (2007) Further characterization of ADAMTS-13 inactivation by thrombin. J Thromb Haemost 5:1010-1018
Lawn R M, Adelman J. Bock S C, Franke A E, Houck C M, Najarian R C, Seeburg P H, Wion K L (1981) The sequence of human serum albumin cDNA and its expression in E. coli. Nucleic Acids Res. 25:6103-6114
M Gibaldi and D Perron, (1982) published by Marcel Dekker, 2nd Rev. edition
Matsumoto C. Hayashi T. Kitada K. Yamashita C. Miyamura M. Mori T. Ukimura A, Ohkita M. Jin O. Takai S. Miyazaki M. Okada Y, Kitaura Y, Matsumura Y. (2009) Chymase plays an important role in left ventricular remodeling induced by intermittent hypoxia in mice. Hypertension. 54:164-171
McGuane J T, Parry L J. (2005) Relaxin and the extracellularmatrix: Molecular mechanisms of action and implications for cardiovascular disease. Expert. Rev. Mol. Med. 7:1-18
Meloun B. Moravek L. Kostka V. (1975) Complete amino acid sequence of human serum albumin. FEBS Lett. 58:134-137
Metra M. Teerlink J R, Felker G M, Greenberg B H, Filippatos G. Ponikowski P. Teichman S L, Unemori E, Voors A A, Weatherley B D, Cotter G. (2010) Dyspnoea and worsening heart failure in patients with acute heart failure: results from the Pre-RELAX-AHF study. Eur J Heart Fail. 12:1130-1139
Minghetti P P, Ruffner D E, Kuang W J, Dennison O E, Hawkins J W, Beattie W G, Dugaiczyk A. (1986) Molecular structure of the human albumin gene is revealed by nucleotide sequence within q11-22 of chromosome 4. J Biol. Chem. 261:6747-6757.
Moore X L, Tan S L, Lo C Y, Fang L. Su Y D, Gao X M, Woodcock E A, Summers R J, Tregear G W, Bathgate R A, Du X J. (2007) Relaxin antagonizes hypertrophy and apoptosis in neonatal rat cardiomyocytes. Endocrinology 148:1582-1589
Morrissey, J. H. (2004) Coagulation factor Vila. In Handbook of Proteolytic Enzymes, 2 edn (Barrett, A. J., Rawlings, N. D. & Woessner, J. F. eds), p. 1659-1662, Elsevier, London
Mortensen, S. B., Sottrup-Jensen, L., Hansen, H. F., Petersen, T. E. and Magnusson, S. (1981) Primary and secondary cleavage sites in the bait region of alpha2-macroglobulin. FEBS Lett 135:295-300
Moss, M. L. and Rasmussen, F. H. (2007) Fluorescent substrates for the proteinases ADAM17, ADAM10, ADAM8, and ADAM12 useful for high-throughput inhibitor screening. Anal Biochem 366:144-148
Nistri, S., Chiappini, L., Sassoli, C. and Bani, D. (2003) Relaxin inhibits lipopolysaccharide-induced adhesion of neutrophils to coronary endothelial cells by a nitric oxide-mediated mechanism. FASEB J. 17:2109-2111
Overall C M. (2004) Dilating the degradome: matrix metalloproteinase 2 (MMP-2) cuts to the heart of the matter. Biochem J. 383(Pt. 3):e5-7
Pasut and Veronese (2009) PEGylation for improving the effectiveness of therapeutic biomolecules. Drugs Today 45:687-695
Pawar, S. C., Demetriou, M. C., Nagle, R. B., Bowden, G. T. and Cress, A. E. (2007) Integrin alpha6 cleavage: a novel modification to modulate cell migration. Exp Cell Res 313:1080-1089
Perna A M, Masini E. Nistri S. Briganti V. Chiappini L. Stefano P. Bigazzi M. Pieroni C. Bani Sacchi T. Bani D. (2005) Novel drug development opportunity for relaxin in acute myocardial infarction: evidences from a swine model. FASEB J. 19:1525-1527 Peters et al, Pharmacokinete analysis: A Practical Approach (1996)
Piedras-Renteria E S, Sherwood O D, Best P M. (1997) Effects of relaxin on rat atrial myocytes. II. Increased calcium influx derived from action potential prolongation. Am J. Physiol. 1997 272(4 Pt 2):H1798-803
Kaufman R J, Sharp P A. (1982) Amplification and expression of sequences cotransfected with a modular dihydrofolate reductase complementary dna gene. J Mol. Biol. 159:601-621.
Radestock Y, Hoang-Vu C. Hombach-Klonisch S. (2008) Relaxin reduces xenograft tumour growth of human MDA-MB-231 breast cancer cells. Breast Cancer Res. 10:R71
Raymond, W. W., Ruggles, S. W., Craik, C. S. & Caughey, G. H. (2003) Albumin is a substrate of human chymase. Prediction by combinatorial peptide screening and development of a selective inhibitor based on the albumin cleavage site. J Biol Chem 278:34517-34524
Robbins, K. C., Summaria, L., Hsieh, B. and Shah, R. J. (1967) The peptide chains of human plasmin. Mechanism of activation of human plasminogen to plasmin. J Biol Chem 242:2333-2342
Rodriguez-Manzaneque, J. C., Westling, J., That, S. N., Luque, A., Knauper, V., Murphy, G., Sandy, J. D. and Iruela-Arispe, M. L. (2002) ADAMTS1 cleaves aggrecan at multiple sites and is differentially inhibited by metalloproteinase inhibitors. Biochem Biophys Res Commun 293:501-508
Roitt and Delves (2004): Essential Immunology (8th Edition, Black-well)
Rosmann, S., Hahn, D., Lottaz, D., Kruse, M. N., Stocker, W. and Sterchi, E. E. (2002) Activation of human meprin-alpha in a cell culture model of colorectal cancer is triggered by the plasminogen-activating system. J Biol Chem 277:40650-40658
Safa, O., Morrissey, J. H., Esmon, C. T. and Esmon, N. L. (1999) Factor VIlanissue factor generates a form of factor V with unchanged specific activity, resistance to activation by thrombin, and increased sensitivity to activated protein C. Biochemistry 38:1829-1837
Sambrook, Fritsch and Maniatis (eds.), Molecular Cloning; A Laboratory Manual, Second Edition, Cold Spring Harbor, N. Y., (1989)
Santora K, Rasa C, Visco D, Steinetz B G, Bagnell C A. (2007) Antiarthritic effects of relaxin, in combination with estrogen, in rat adjuvant induced arthritis. J. Pharmacol. Exp. Ther. 322:887-893
Schlechter and Berger (1967) On the size of the active site in proteases. I. Papain. Biochem Biophys Res Commun 27:157-162
Schlechter and Berger (1968) On the active site of proteases. 3. Mapping the active site of papain; specific peptide inhibitors of papain. Biochem Biophys Res Commun 32:898-902
Schmid S R, (2009) Fusion-proteins as biopharmaceuticals—applications and challenges. Curr Opin Drug Discov Devel. 12:284-95
Schwabe C. Büllesbach E E. (2007) Relaxin, the relaxin-like factor and their receptors. Adv Exp Med. Biol. 612:14-25
Shaw J A, Delday M I, Hart A W, Docherty H M, Maltin C A, Docherty K. (2002) Secretion of bioactive human insulin following plasmid-mediated gene transfer to non-neuroendocrine cell lines, primary cultures and rat skeletal muscle in vivo. J. Endocrinol. 172:653-672
Sim, R. B. and Tsiftsoglou, S. A. (2004) Proteases of the complement system. Biochem Soc Trans 32:21-27
Starck, S., Van Den Steen, P. E., Verbeek, R., van Noort, J. M. and Opdenakker, G. (2003) A novel rationale for inhibition of gelatinase B in multiple sclerosis: MMP-9 destroys alphaB-crystallin and generates a promiscuous T cell epitope. J Neuroimmunol 141:47-57
Suzuki, F., Murakami, K., Nakamura, Y. & Inagami, T. Renin. In Handbook of Proteolytic Enzymes, 2 edn (Barrett, A. J., Rawlings, N. D. & Woessner, J. F. eds), p. 54-61, Elsevier, London (2004)
Szmola, R. and Sahin-Toth, M. (2007) Chymotrypsin C (caldecrin) promotes degradation of human cationic trypsin: identity with Rinderknecht's enzyme Y. Proc Natl Acad Sci USA 104:11227-11232
Taggart, C. C., Lowe, G. J., Greene, C. M., Mulgrew, A. T., O'Neill, S. J., Levine, R. L. and McElvaney, N. G. (2001) Cathepsin B. L. and S cleave and inactivate secretory leucoprotease inhibitor. J Biol Chem 276:33345-33352
Teerlink J R, Metra M, Felker G M, Ponikowski P, Voors A A, Weatherley B D, Marmor A, Katz A, Grzybowski J, Unemori E, Teichman S L, Cotter G. (2009) Relaxin for the treatment of patients with acute heart failure (Pre-RELAX-AHF): a multicentre, randomised, placebo-controlled, parallel-group, dose-finding phase IIb study. Lancet. 373:1429-39
Tortorella, M. D., Arner, E. C., Hills, R., Gormley, J., Fok, K., Pegg, L., Munie, G. and Malfait, A. M. (2005) ADAMTS-4 (aggrecanase-1): N-terminal activation mechanisms. Arch Biochem Biophys 444:34-44
Toth, M., Taskinen, P., and Ruskoaho, H. (1996). Relaxin stimulates atrial natriuretic peptide secretion in perfused rat heart. J Endocrinol 150: 487-495
Ueno, T., Linder, S., Na, C. L., Rice, W. R., Johansson, J. and Weaver, T. E. (2004) Processing of pulmonary surfactant protein B by napsin and cathepsin H. J Biol Chem 279:16178-16184
Urlaub G. Chasin L A. (1980) Isolation of Chinese hamster cell mutants deficient in dihydrofolate reductase activity. Proc Natl Acad Sci USA. 77:4216-4220
Vakili, J., Standker, L., Detheux, M., Vassart, G., Forssmann, W. G. and Parmentier, M. (2001) Urokinase plasminogen activator and plasmin efficiently convert hemofiltrate C C chemokine 1 into its active [9-74] processed variant. J Immunol 167:3406-3413
Walter, M., Sutton, R. M. & Schechter, N. M. (1999) Highly efficient inhibition of human chymase by alpha(2)-macroglobulin. Arch Biochem Biophys 368:276-284
Wang P. Li H W, Wang Y P, Chen H. Zhang P. (2009) Effects of recombinant human relaxin upon proliferation of cardiac fibroblast and synthesis of collagen under high glucose condition. J Endocrinol Invest. 32:242-247
Wilkinson, T. N., Speed, T. P., Tregear, G. W., Bathgate, R. A. (2005). Evolution of the relaxin-like peptide family. BMC Evol Biol 5:14
Witt, H., Luck, W., Hennies, H. C., Classen, M., Kage, A., Lass, U., Landt, O. and Becker, M. (2000) Mutations in the gene encoding the serine protease inhibitor, Kazal type 1 are associated with chronic pancreatitis. Nat Genet. 25:213-216
Zhang J, Qi Y F, Geng B. Pan C S, Zhao J, Chen L, Yang J, Chang J K, Tang C S. (2005) Effect of relaxin on myocardial ischemia injury induced by isoproterenol. Peptides 26:1632-1639
EP0322094
EP0399666
EP0413622
U.S. Pat. Nos. 4,399,216, 4,634,665
U.S. Pat. No. 4,510,245 by Bell et al
U.S. Pat. No. 4,968,615 by Schaffner et al.
U.S. Pat. No. 5,168,062 by Stinski
U.S. Pat. No. 5,179,017, by Axel et al.
U.S. Pat. No. 4,657,760
U.S. Pat. No. 4,816,397 by Boss et al.
U.S. Pat. No. 5,225,212.
U.S. Pat. No. 5,206,344
U.S. Pat. No. 5,525,491
U.S. Pat. No. 7,271,149
U.S. Pat. No. 4,683,195
U.S. Pat. No. 4,683,195
U.S. Pat. No. US2011/0130332
US20100104588
WO 00/06568
WO 01/19355
WO 01/19778
WO 01/77137
WO 02/070462
WO 02/42301
WO 93/15199
WO 93/15200
WO 97/26265
WO01/45746
WO2001/058957
WO2010/054699
WO93/15199

1. A fusion protein comprising Relaxin-PCS-HEM, HEM-PCS-Relaxin, proRelaxin-PCS-HEM, or HEM-PCS-proRelaxin wherein
Relaxin comprises a Relaxin A chain polypeptide or a functional variant thereof, optionally a Relaxin C-Chain polypeptide,
and a Relaxin B chain polypeptide or a functional variant thereof,
PCS comprises an endo-protease cleavage site, and
HEM is a proteinaceous half-life extending moiety.

2. (canceled)

3. A fusion protein or polypeptide according to claim 1, wherein the PCS is a cleavage site of an extracellular endo-protease.

4. A fusion protein or polypeptide according to claim 3, wherein the extracellular endo-protease is an endogenous endo-protease.

5. A fusion polypeptide according to claim 1, wherein the proteinaceous half-life extending moieties are comprised in a group of proteinaceous half-life extending moieties consisting of immunoglobulin Fc domain, serum albumin, transferrin and serum albumin binding protein.

6. A fusion protein according to claim 1, wherein the Relaxin A chain is human Relaxin 2 A chain and the Relaxin B chain is human Relaxin 2 B chain.

7. A polynucleotide encoding a fusion protein according to claim 1.

8. A vector comprising a polynucleotide according to claim 7.

9. A host cell comprising polynucleotide according to claim 7.

10. A method of producing a fusion protein comprising the steps of cultivating a host cell of claim 9 further comprising a prohormone convertase activity and isolating the fusion protein.

11. A pharmaceutical composition comprising a fusion protein according to claim 1.

12-13. (canceled)

14. A method of treating a cardiovascular disease, lung disease, fibrotic disorder or kidney disease comprising the administration of a therapeutically effective dose of a fusion protein according to claim 1.

15. A treatment according to claim 14, wherein the cardiovascular disease is comprised in the group of coronary heart disease, acute coronary syndrome, heart failure, or myocardial infarction.


SEQUENCE LISTING (32 sequences)

SEQ ID NO: 1 - protein, 397 aa, Artificial Sequence
  Relaxin-Fxa cleavage site-hum IgG1 Fc
SWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFCIEGRMDPKACDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 679 aa, Homo Sapiens
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 3 - protein, 585 aa, Homo Sapiens [sequence omitted]

SEQ ID NO: 4 - protein, 231 aa, Homo Sapiens
PKACDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 160 aa, Homo Sapiens
SWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFC

SEQ ID NO: 6 - protein, 24 aa, Homo Sapiens
QLYSALANKCCHVGCTKRSLARFC

SEQ ID NO: 7 - protein, 16 aa, Homo Sapiens
KCCHVGCTKRSLARFC

SEQ ID NO: 8 - protein, 28 aa, Homo Sapiens
SWMEEVIKLCGRELVRAQIAICGMSTWS

SEQ ID NO: 9 - protein, 117 aa, Homo Sapiens
RAAPYGVRLCGREFIRAVIFTCGGSRWRRSDILAHEAMGDTFPDADADEDSLAGELDEAMGSSEWLALTKSPQAFYRGRPSWQGTPGVLRGSRDVLAGLSSSCCKWGCSKSEISSLC

SEQ ID NO: 10 - protein, 24 aa, Homo Sapiens
DVLAGLSSSCCKWGCSKSEISSLC

SEQ ID NO: 11 - protein, 27 aa, Homo Sapiens
RAAPYGVRLCGREFIRAVIFTCGGSRW

SEQ ID NO: 12 - protein, 15 aa, Homo Sapiens
CCKWGCSKSEISSLC

SEQ ID NO: 13 - protein, 399 aa, Artificial Sequence
  Relaxin - MMP9 cleavage site - humFc IgG1
SWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFCRAKRFASLPKACDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 399 aa, Artificial Sequence
  Relaxin - Chymase cleavage site- humFc IgG1
SWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFCRVGFYESDPKACDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 399 aa, Artificial Sequence
  Relaxin - Trypsin cleavage site- humFc IgG1
SWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFCINARVSTIPKACDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 399 aa, Artificial Sequence [sequence omitted]

SEQ ID NO: 17 - DNA, 1191 bp, Artificial Sequence
  Relaxin - Fxa cleavage site- hum IgG1 Fc
agctggatggaagaagtgattaaactgtgcggccgcgaactggtgcgcgcgcagattgcgatttgcggcatgagcacctggagcaaacgcagcctgagccaggaagatgcgccgcagaccccgcgcccggtggcggaaattgtgccgagctttattaacaaagataccgaaaccattaacatgatgagcgaatttgtggcgaacctgccgcaggaactgaaactgaccctgagcgaaatgcagccggcgctgccgcagctgcagcagcatgtgccggtgctgaaagatagcagcctgctgtttgaagaatttaaaaaactgattcgcaaccgccagagcgaagcggcggatagcagcccgagcgaactgaaatatctgggcctggatacccatagccgcaaaaaacgccagctgtatagcgcgctggcgaacaaatgctgccatgtgggctgcaccaaacgcagcctggcgcgcttttgcattgaaggccgcatggatccgaaagcgtgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 18 - DNA, 2037 bp, Homo Sapiens
gtgccggataaaaccgtgcgctggtgcgcggtgagcgaacatgaagcgaccaaatgccagagctttcgcgatcatatgaaaagcgtgattccgagcgatggcccgagcgtggcgtgcgtgaaaaaagcgagctatctggattgcattcgcgcgattgcggcgaacgaagcggatgcggtgaccctggatgcgggcctggtgtatgatgcgtatctggcgccgaacaacctgaaaccggtggtggcggaattttatggcagcaaagaagatccgcagaccttttattatgcggtggcggtggtgaaaaaagatagcggctttcagatgaaccagctgcgcggcaaaaaaagctgccataccggcctgggccgcagcgcgggctggaacattccgattggcctgctgtattgcgatctgccggaaccgcgcaaaccgctggaaaaagcggtggcgaacttttttagcggcagctgcgcgccgtgcgcggatggcaccgattttccgcagctgtgccagctgtgcccgggctgcggctgcagcaccctgaaccagtattttggctatagcggcgcgtttaaatgcctgaaagatggcgcgggcgatgtggcgtttgtgaaacatagcaccatttttgaaaacctggcgaacaaagcggatcgcgatcagtatgaactgctgtgcctggataacacccgcaaaccggtggatgaatataaagattgccatctggcgcaggtgccgagccataccgtggtggcgcgcagcatgggcggcaaagaagatctgatttgggaactgctgaaccaggcgcaggaacattttggcaaagataaaagcaaagaatttcagctgtttagcagcccgcatggcaaagatctgctgtttaaagatagcgcgcatggctttctgaaagtgccgccgcgcatggatgcgaaaatgtatctgggctatgaatatgtgaccgcgattcgcaacctgcgcgaaggcacctgcccggaagcgccgaccgatgaatgcaaaccggtgaaatggtgcgcgctgagccatcatgaacgcctgaaatgcgatgaatggagcgtgaacagcgtgggcaaaattgaatgcgtgagcgcggaaaccaccgaagattgcattgcgaaaattatgaacggcgaagcggatgcgatgagcctggatggcggctttgtgtatattgcgggcaaatgcggcctggtgccggtgctggcggaaaactataacaaaagcgataactgcgaagataccccggaagcgggctattttgcggtggcggtggtgaaaaaaagcgcgagcgatctgacctgggataacctgaaaggcaaaaaaagctgccataccgcggtgggccgcaccgcgggctggaacattccgatgggcctgctgtataacaaaattaaccattgccgctttgatgaattttttagcgaaggctgcgcgccgggcagcaaaaaagatagcagcctgtgcaaactgtgcatgggcagcggcctgaacctgtgcgaaccgaacaacaaagaaggctattatggctataccggcgcgtttcgctgcctggtggaaaaaggcgatgtggcgtttgtgaaacatcagaccgtgccgcagaacaccggcggcaaaaacccggatccgtgggcgaaaaacctgaacgaaaaagattatgaactgctgtgcctggatggcacccgcaaaccggtggaagaatatgcgaactgccatctggcgcgcgcgccgaaccatgcggtggtgacccgcaaagataaagaagcgtgcgtgcataaaattctgcgccagcagcagcatctgtttggcagcaacgtgaccgattgcagcggcaacttttgcctgtttcgcagcgaaaccaaagatctgctgtttcgcgatgataccgtgtgcctggcgaaactgcatgatcgcaacacctatgaaaaatatctgggcgaagaatatgtgaaagcggtgggcaacctgcgcaaatgcagcaccagcagcctgctggaagcgtgcacctttcgccgcccg

SEQ ID NO: 19 - DNA, 1755 bp, Homo Sapiens
gatgcgcataaaagcgaagtggcgcatcgctttaaagatctgggcgaagaaaactttaaagcgctggtgctgattgcgtttgcgcagtatctgcagcagtgcccgtttgaagatcatgtgaaactggtgaacgaagtgaccgaatttgcgaaaacctgcgtggcggatgaaagcgcggaaaactgcgataaaagcctgcataccctgtttggcgataaactgtgcaccgtggcgaccctgcgcgaaacctatggcgaaatggcggattgctgcgcgaaacaggaaccggaacgcaacgaatgctttctgcagcataaagatgataacccgaacctgccgcgcctggtgcgcccggaagtggatgtgatgtgcaccgcgtttcatgataacgaagaaacctttctgaaaaaatatctgtatgaaattgcgcgccgccatccgtatttttatgcgccggaactgctgttttttgcgaaacgctataaagcggcgtttaccgaatgctgccaggcggcggataaagcggcgtgcctgctgccgaaactggatgaactgcgcgatgaaggcaaagcgagcagcgcgaaacagcgcctgaaatgcgcgagcctgcagaaatttggcgaacgcgcgtttaaagcgtgggcggtggcgcgcctgagccagcgctttccgaaagcggaatttgcggaagtgagcaaactggtgaccgatctgaccaaagtgcataccgaatgctgccatggcgatctgctggaatgcgcggatgatcgcgcggatctggcgaaatatatttgcgaaaaccaggatagcattagcagcaaactgaaagaatgctgcgaaaaaccgctgctggaaaaaagccattgcattgcggaagtggaaaacgatgaaatgccggcggatctgccgagcctggcggcggattttgtggaaagcaaagatgtgtgcaaaaactatgcggaagcgaaagatgtgtttctgggcatgtttctgtatgaatatgcgcgccgccatccggattatagcgtggtgctgctgctgcgcctggcgaaaacctatgaaaccaccctggaaaaatgctgcgcggcggcggatccgcatgaatgctatgcgaaagtgtttgatgaatttaaaccgctggtggaagaaccgcagaacctgattaaacagaactgcgaactgtttgaacagctgggcgaatataaatttcagaacgcgctgctggtgcgctataccaaaaaagtgccgcaggtgagcaccccgaccctggtggaagtgagccgcaacctgggcaaagtgggcagcaaatgctgcaaacatccggaagcgaaacgcatgccgtgcgcggaagattatctgagcgtggtgctgaaccagctgtgcgtgctgcatgaaaaaaccccggtgagcgatcgcgtgaccaaatgctgcaccgaaagcctggtgaaccgccgcccgtgctttagcgcgctggaagtggatgaaacctatgtgccgaaagaatttaacgcggaaacctttacctttcatgcggatatttgcaccctgagcgaaaaagaacgccagattaaaaaacagaccgcgctggtggaactggtgaaacataaaccgaaagcgaccaaagaacagctgaaagcggtgatggatgattttgcggcgtttgtggaaaaatgctgcaaagcggatgataaagaaacctgctttgcggaagaaggcaaaaaactggtggcggcgagccaggcggcgctgggcctg

SEQ ID NO: 20 - DNA, 693 bp, Homo Sapiens
ccgaaagcgtgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 21 - DNA, 480 bp, Homo Sapiens
agctggatggaagaagtgattaaactgtgcggccgcgaactggtgcgcgcgcagattgcgatttgcggcatgagcacctggagcaaacgcagcctgagccaggaagatgcgccgcagaccccgcgcccggtggcggaaattgtgccgagctttattaacaaagataccgaaaccattaacatgatgagcgaatttgtggcgaacctgccgcaggaactgaaactgaccctgagcgaaatgcagccggcgctgccgcagctgcagcagcatgtgccggtgctgaaagatagcagcctgctgtttgaagaatttaaaaaactgattcgcaaccgccagagcgaagcggcggatagcagcccgagcgaactgaaatatctgggcctggatacccatagccgcaaaaaacgccagctgtatagcgcgctggcgaacaaatgctgccatgtgggctgcaccaaacgcagcctggcgcgcttttgc

SEQ ID NO: 22 - DNA, 72 bp, Homo Sapiens
cagctgtatagcgcgctggcgaacaaatgctgccatgtgggctgcaccaaacgcagcctggcgcgcttttgc

SEQ ID NO: 23 - DNA, 48 bp, Homo Sapiens
aaatgctgccatgtgggctgcaccaaacgcagcctggcgcgcttttgc

SEQ ID NO: 24 - DNA, 84 bp, Homo Sapiens
agctggatggaagaagtgattaaactgtgcggccgcgaactggtgcgcgcgcagattgcgatttgcggcatgagcacctggagc

SEQ ID NO: 25 - DNA, 351 bp, Homo Sapiens
cgcgcggcgccgtatggcgtgcgcctgtgcggccgcgaatttattcgcgcggtgatttttacctgcggcggcagccgctggcgccgcagcgatattctggcgcatgaagcgatgggcgatacctttccggatgcggatgcggatgaagatagcctggcgggcgaactggatgaagcgatgggcagcagcgaatggctggcgctgaccaaaagcccgcaggcgttttatcgcggccgcccgagctggcagggcaccccgggcgtgctgcgcggcagccgcgatgtgctggcgggcctgagcagcagctgctgcaaatggggctgcagcaaaagcgaaattagcagcctgtgc

SEQ ID NO: 26 - DNA, 72 bp, Homo Sapiens
gatgtgctggcgggcctgagcagcagctgctgcaaatggggctgcagcaaaagcgaaattagcagcctgtgc

SEQ ID NO: 27 - DNA, 81 bp, Homo Sapiens
cgcgcggcgccgtatggcgtgcgcctgtgcggccgcgaatttattcgcgcggtgatttttacctgcggcggcagccgctgg

SEQ ID NO: 28 - DNA, 45 bp, Homo Sapiens
tgctgcaaatggggctgcagcaaaagcgaaattagcagcctgtgc

SEQ ID NO: 29 - DNA, 1197 bp, Artificial Sequence
  Relaxin - MMP9 cleavage site- humFc IgG1
agctggatggaagaagtgattaaactgtgcggccgcgaactggtgcgcgcgcagattgcgatttgcggcatgagcacctggagcaaacgcagcctgagccaggaagatgcgccgcagaccccgcgcccggtggcggaaattgtgccgagctttattaacaaagataccgaaaccattaacatgatgagcgaatttgtggcgaacctgccgcaggaactgaaactgaccctgagcgaaatgcagccggcgctgccgcagctgcagcagcatgtgccggtgctgaaagatagcagcctgctgtttgaagaatttaaaaaactgattcgcaaccgccagagcgaagcggcggatagcagcccgagcgaactgaaatatctgggcctggatacccatagccgcaaaaaacgccagctgtatagcgcgctggcgaacaaatgctgccatgtgggctgcaccaaacgcagcctggcgcgcttttgccgcgcgaaacgctttgcgagcctgccgaaagcgtgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 30 - DNA, 1197 bp, Artificial Sequence
  Relaxin - Chymase cleavage site- humFc IgG1
agctggatggaagaagtgattaaactgtgcggccgcgaactggtgcgcgcgcagattgcgatttgcggcatgagcacctggagcaaacgcagcctgagccaggaagatgcgccgcagaccccgcgcccggtggcggaaattgtgccgagctttattaacaaagataccgaaaccattaacatgatgagcgaatttgtggcgaacctgccgcaggaactgaaactgaccctgagcgaaatgcagccggcgctgccgcagctgcagcagcatgtgccggtgctgaaagatagcagcctgctgtttgaagaatttaaaaaactgattcgcaaccgccagagcgaagcggcggatagcagcccgagcgaactgaaatatctgggcctggatacccatagccgcaaaaaacgccagctgtatagcgcgctggcgaacaaatgctgccatgtgggctgcaccaaacgcagcctggcgcgcttttgccgcgtgggcttttatgaaagcgatccgaaagcgtgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 31 - DNA, 1197 bp, Artificial Sequence
  Relaxin - Trypsin cleavage site- humFc IgG1
agctggatggaagaagtgattaaactgtgcggccgcgaactggtgcgcgcgcagattgcgatttgcggcatgagcacctggagcaaacgcagcctgagccaggaagatgcgccgcagaccccgcgcccggtggcggaaattgtgccgagctttattaacaaagataccgaaaccattaacatgatgagcgaatttgtggcgaacctgccgcaggaactgaaactgaccctgagcgaaatgcagccggcgctgccgcagctgcagcagcatgtgccggtgctgaaagatagcagcctgctgtttgaagaatttaaaaaactgattcgcaaccgccagagcgaagcggcggatagcagcccgagcgaactgaaatatctgggcctggatacccatagccgcaaaaaacgccagctgtatagcgcgctggcgaacaaatgctgccatgtgggctgcaccaaacgcagcctggcgcgcttttgcattaacgcgcgcgtgagcaccattccgaaagcgtgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 32 - DNA, 1197 bp, Artificial Sequence
  Relaxin - Elastase cleavage site- humFc IgG1
agctggatggaagaagtgattaaactgtgcggccgcgaactggtgcgcgcgcagattgcgatttgcggcatgagcacctggagcaaacgcagcctgagccaggaagatgcgccgcagaccccgcgcccggtggcggaaattgtgccgagctttattaacaaagataccgaaaccattaacatgatgagcgaatttgtggcgaacctgccgcaggaactgaaactgaccctgagcgaaatgcagccggcgctgccgcagctgcagcagcatgtgccggtgctgaaagatagcagcctgctgtttgaagaatttaaaaaactgattcgcaaccgccagagcgaagcggcggatagcagcccgagcgaactgaaatatctgggcctggatacccatagccgcaaaaaacgccagctgtatagcgcgctggcgaacaaatgctgccatgtgggctgcaccaaacgcagcctggcgcgcttttgcggcctgcgcgtgggcttttatgaaccgaaagcgtgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa